SEQUENCE LISTING (7 sequences)

SEQ ID NO: 1 - DNA, 523 bp, Homo sapiens
ctcctggttcaaaagcagctaaaccaaaagaagcctccagacagccctgagatcacctaaaaagctgctaccaagacagccacgaagatcctaccaaaatgaagcgcttcctcttcctcctactcaccatcagcctcctggttatggtacagatacaaactggactctcaggacaaaacgacaccagccaaaccagcagcccctcagcatccagcaacataagcggaggcattttccttttcttcgtggccaatgccataatccacctcttctgcttcagttgaggtgacacgtctcagccttagccctgtgccccctgaaacagctgccaccatcactcgcaagagaatcccctccatctttgggaggggttgatgccagacatcaccaggttgtagaagttgacaggcagtgccatgggggcaacagccaaaataggggggtaatgatgtaggggccaagcagtgcccagctgggggtcaataaagttacccttgtacttgcaaaaaaaaaaaaaaaaaaa

SEQ ID NO: 2 - protein, 61 aa, Homo sapiens
MKRFLFLLLTISLLVMVQIQTGLSGQNDTSQTSSPSASSNISGGIFLFFVANAIIHLFCFS

SEQ ID NO: 3 - protein, 12 aa, Homo sapiens
GQNDTSQTSSPS

SEQ ID NO: 4 - protein, 11 aa, Pan troglodytes
SQNATSQSSPS

SEQ ID NO: 5 - protein, 24 aa, Mus musculus
GQATTAASGTNKNSTSTKKTPLKS

SEQ ID NO: 6 - protein, 46 aa, Rattus norvegicus
GQNSTAVTTPANKAATTAAATTKAAATTATKTTTAVRKTPGKPPKA

SEQ ID NO: 7 - protein, 19 aa, Canis familiaris
GNSTTPRMTTKKVKSATPA

(19) United States
(12) Patent Application Publication
(10) Pub. No.: US 2022/0185864 A1
(43) Pub. Date: Jun. 16, 2022

(54) IMMUNOSUPPRESSIVE GLYCOFORMS OF SOLUBLE CD52

(71) Applicants: THE WALTER AND ELIZA HALL INSTITUTE OF MEDICAL RESEARCH, Parkville (AU); MACQUARIE UNIVERSITY, North Ryde (AU)

(72) Inventors: Leonard Charles HARRISON, Parkville (AU); Esther BANDALA- SANCHEZ, Parkville (AU); Ethan David GODDARD-BORGER, Parkville (AU); Nicolle Hannah PACKER, North Ryde (AU); Shathili Abdulrahman MANSOUR, North Ryde (AU)

(21) Appl. No.: 17/436,342

(22) PCT Filed: Mar. 7, 2019
(86) PCT No.: PCT/AU2019/050197

§ 371 Date: Sep. 3, 2021

(51) Int. Cl. C07K 14/705; A61K 38/17; A61K 35/16; A61K 38/28

(52) U.S. Cl. CPC C07K 14/70503; A61K 38/1774; C07K 2319/30; A61K 38/28; A61K 35/16

(57) ABSTRACT

The present disclosure relates to glycoprotein CD52 and fusion proteins thereof, wherein the CD52 glycoprotein has α-2,3-sialylated N-glycans, O-glycosylation and a pI of about 5 to about 6. The disclosure further relates to the preparation and purification of these proteins and their use in the suppression of effector T-cell function and/or immune response, such as in the treatment of diseases or conditions mediated by effector T-cell function.

### FIELD OF THE INVENTION

The present disclosure relates to CD52 glycoproteins, in particular soluble CD52 glycoproteins, and their fusion proteins, and the use of CD52 glycoproteins and their fusion proteins in the suppression of effector T-cell function and/or immune response, and in the treatment of diseases or conditions mediated by effector T-cell function. The disclosure further relates to the preparation and purification of CD52 glycoproteins and fusion proteins.

### BACKGROUND OF THE INVENTION

Human soluble CD52 is a glycoprotein composed of only 12 amino acids. There are analogues in other mammals. Soluble CD52 is released from the surface of activated T cells and initiates immunosuppression by first sequestering the pro-inflammatory damage-associated molecular pattern (DAMP) protein, high-mobility group box 1 (HMGB1), followed by binding to the inhibitory sialic acid-binding immunoglobulin-like lectin-10 (Siglec-10).

CD52 glycoproteins are modified by N-linked and/or O-linked glycosylation. The glycosylation profile of the CD52 responsible for this binding and the related activity is yet to be determined.

Mass spectroscopic (MS) analysis has shown the N-glycans on human leukocyte CD52 exhibit extensive heterogeneity with multi-antennary complexes containing core α-1,6 fucosylation, abundant polyLacNAc extensions and variable sialylation. Although one or more CD52 N-glycans are known to be required for bioactivity, the structure of the one or more active CD52 N-glycans has not been fully elucidated. In addition, despite the six potential amino acid sites suitable for O-glycosylation, O-glycosylation of CD52 has not been analysed.

There is a need to determine the bioactive CD52 glycoforms. Identification of the bioactive CD52 glycoforms or a group of glycoforms with better bioactivity, could allow for development of an immune suppressor, with improved efficacy, efficiency and/or more consistency in its effect.

### SUMMARY OF THE INVENTION

The present inventors have identified glycoforms of soluble CD52 glycoprotein that suppress effector T-cell function and/or immune response.

In a first aspect, the present invention provides one or more soluble CD52 glycoproteins, wherein the one or more soluble CD52 glycoproteins comprise:
(i) one or more multi-antennary N-linked α-2,3-sialylated glycans, preferably tetra-antennary N-linked α-2,3-sialylated glycans; and
(ii) an isoelectric point (pl) of about 5 to about 6.

Optionally, the soluble CD52 glycoprotein further comprises one or more O-glycans, preferably one or more O-glycans, preferably di-sialylated O-glycans, and/or no bisecting GlcNAc structures on the N-glycans.

In an alternative first aspect, the present invention provides one or more soluble CD52 glycoproteins, wherein the one or more soluble CD52 glycoproteins comprise:
(i) one or more multi-antennary N-linked α-2,3-sialylated glycans, preferably tetra-antennary N-linked α-2,3-sialylated glycans; and
(ii) no N-linked bisecting GlcNAc structures.

Optionally, the soluble CD52 glycoprotein further comprises one or more O-glycans, preferably one or more O-glycans, preferably di-sialylated O-glycans, and/or a pl of about 5 to about 6.

In an alternative first aspect, the present invention provides one or more soluble CD52 glycoproteins, wherein the one or more soluble CD52 glycoproteins comprise:
(iii) one or more N-linked α-2,3-sialylated glycans;
(iv) one or more O-glycans, preferably one or more di-sialylated O-glycans; and
(v) no N-linked bisecting GlcNAc structures.

Optionally, the soluble CD52 glycoprotein further comprises one or more multi-antennary sialylated N-glycans, preferably tetra-antennary sialylated N-glycans and/or a pl of about 5 to about 6.

In an alternative first aspect, the present invention provides one or more soluble CD52 glycoproteins, wherein the one or more soluble CD52 glycoproteins comprise:
(iii) one or more N-linked α-2,3-sialylated glycans;
(iv) one or more O-glycans, preferably one or more di-sialylated O-glycans; and
(v) a pl of about 5 to about 6.

Optionally, the soluble CD52 glycoprotein further comprises one or more multi-antennary sialylated N-glycans, preferably tetra-antennary sialylated N-glycans and/or no N-linked bisecting GlcNAc structures.

In a further alternative first aspect, the present invention provides one or more soluble CD52 glycoproteins, wherein the one or more soluble CD52 glycoproteins comprises:
(i) one or more multi-antennary N-linked α-2,3-sialylated glycans; and
(ii) one or more di-sialylated O-glycans.

Optionally, this aspect also has a pl of about 5 to about 6 and/or no N-linked bisecting GlcNAc structures.

The soluble CD52 glycoprotein of all embodiments of the invention optionally has an amino acid sequence comprising at least one amino acid suitable for N-linked glycosylation and an amino acid sequence at least 60% identical to any one or more of

(SEQ ID NO: 3)
GQNDTSQTSSPS,
(SEQ ID NO: 4)
SQNATSQSSPS,
(SEQ ID NO: 5)
GQATTAASGTNKNSTSTKKTPLKS,
(SEQ ID NO: 6)
GQNSTAVTTPANKAATTAAATTKAAATTATKTTTAVRKTPGKPPKA
and
(SEQ ID NO: 7)
GNSTTPRMTTKKVKSATPA.

Optionally, the soluble CD52 glycoprotein has an amino acid sequence that is at least 65%, at least 70%, at least 75%, at least 80%, at least 85%, at least 90%, at least 95% identical to any one or more of the amino acid sequences identified in SEQ ID NOs: 3, 4, 5, 6 or 7. Alternatively, the soluble CD52 glycoprotein has an amino acid sequence that is identical to any one or more of the amino acid sequences identified in SEQ ID NOs: 3, 4, 5, 6 or 7.

Optionally, the soluble CD52 glycoprotein is human.

Amino acids suitable for N-linked glycosylation are often asparagine residues that are part of an Asn-X-Ser/Thr (ie N—X—S/T) sequon ie an asparagine one amino acid away from either serine or threonine. X can be any amino acid other than proline. Where an N—X—S/T sequon is present, the amino acids on the peptide not in the N—X—S/T sequon can be replaced with another amino acid. It is preferred if the amino acid is replaced with another of similar biochemical properties, eg charge, size, hydrophoboicity.

Therefore, in some embodiments, the soluble CD52 glycoprotein has an N—X—S/T sequon and is at least 65%, at least 70%, at least 75%, at least 80%, at least 85%, at least 90%, or at least 95% identical to any one or more of the amino acid sequences identified in SEQ ID NOs: 3, 4, 5, 6 or 7.

Optionally, the soluble CD52 glycoprotein has an amino acid sequence that is at least 60% identical to SEQ ID NO: 3 and the N-linked glycan is linked to the asparagine residue in that peptide (ie Asn3; N3).

Optionally, the N-linked α-2,3-sialylated glycan is a multi-antennary sialylated N-glycan, a di-, tri- or tetra-antennary sialylated N-glycan, or a tri- or tetra-antennary sialylated N-glycan. Tetra-antennary sialylated N-glycans are preferred. Optionally, the multi-antennary α-2,3-sialylated N-glycan has polyLacNAc extensions.

In all embodiments of the invention, the one or more N-glycans in the CD52 glycoprotein of the invention are optionally selected from the group consisting of:

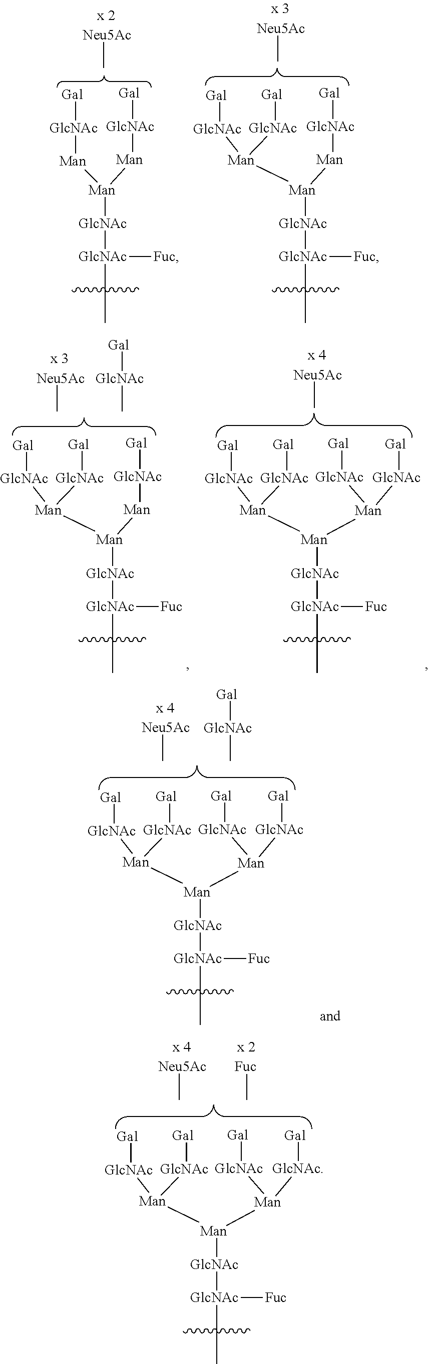

In these and the similar structures throughout this specification, the N-glycan is linked to the peptide through the bond marked with the wavy line. In addition, the glycan residues above the bracket are connected to the glycan below the bracket through any of the upper-most residues below the bracket or through the other residues above the bracket, to a total number of residues as indicated on the structure depicted.

The one or more N-glycans in the CD52 glycoprotein of the invention are optionally selected from the group consisting of:

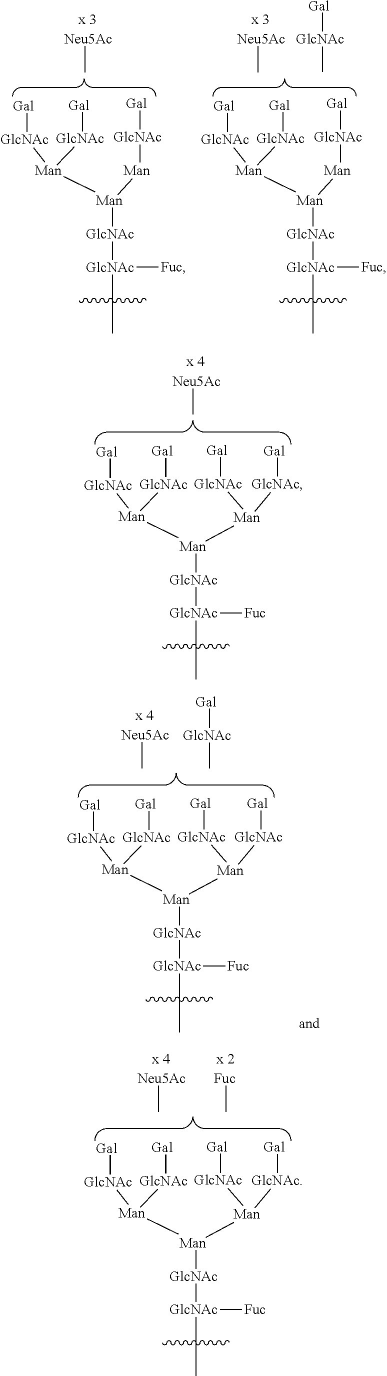

The one or more N-glycan in the CD52 glycoprotein of the invention are optionally selected from the group consisting of:

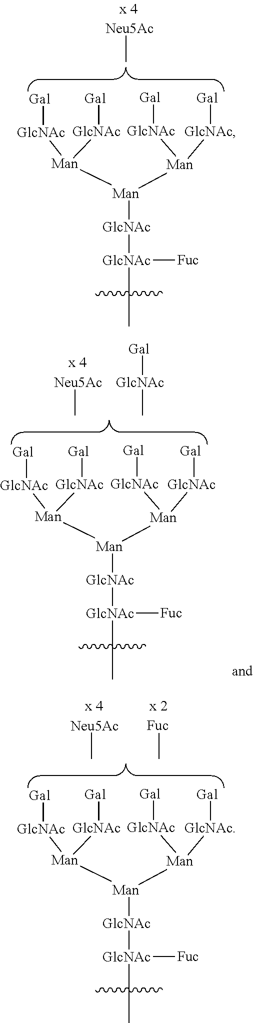

In all embodiments of the invention, the O-glycosylation is optionally sialylated, preferably mono- and/or di-sialylated. Optionally, the soluble CD52 glycoprotein has an amino acid sequence that is at least 60% identical to SEQ ID NO: 3 and the peptide is O-glycosylated at Ser (S) 12, Ser (S) 10 and/or Thr (T) 8, preferably the 0-glycosylation is mono-sialylated at Thr (T) 8 or di-sialylated and at Ser (S) 12 and/or Ser (S) 10.

The one or more O-glycans in the CD52 glycoprotein of the invention are optionally selected from the group consisting of:

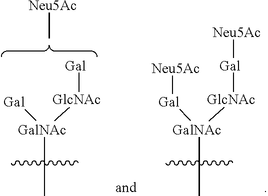

In a second aspect, the present invention provides a fusion protein comprising the soluble CD52 glycoprotein according the first or an alternative first aspect of the invention (or their embodiments) fused with a second protein. Optionally, the second protein comprises an antibody fragment, preferably an Fc or an IgG1 Fc. Optionally, the second protein comprises a purification tag. The purification tag is optionally selected from the group consisting of a His tag, T7 tag, FLAG tag, S-tag, HA tag, c-Myc tag, DHFR, a chitin biding domain, a calmodulin binding domain, a cellulose binding domain and a Strep 2 tag. A Strep 2 tag is preferred.

In some embodiments, the fusion protein comprises a second protein having both an antibody fragment and a purification tag as described above.

The fusion protein optionally has an isoelectric point (pl) of about 5 to about 6.

In an alternative of the first and second aspects of invention, the present invention provides a composition comprising serum and a soluble CD52 glycoprotein or fusion protein according to the first, alternative first or second aspect of the invention (or any of their embodiments).

In an another alternative of the first and second aspects of invention, the present invention provides a composition comprising insulin and/or an autoantigen and a soluble CD52 glycoprotein or fusion protein according to the first, alternative first or second aspect of the invention (or any of their embodiments).

In a third aspect, the present invention provides a composition comprising a plurality of soluble CD52 glycoproteins or their fusion proteins, wherein the CD52 glycoproteins of the composition comprise:
(i) an increased amount of α-2,3-sialylated N-glycans relative to wild-type soluble CD52 glycoprotein;
(ii) an increased amount of tri- and tetra-antennary N-glycans relative to wild-type soluble CD52 glycoprotein; and/or
(iii) an increased amount of O-glycosylation relative to wild-type soluble CD52 glycoprotein;
wherein wild-type soluble CD52 glycoprotein is CD52 from the human spleen.

In an alternative third aspect, the present invention provides a composition comprising a plurality of soluble CD52 glycoproteins or their fusion proteins, wherein the CD52 glycoproteins of the composition comprise:
(i) an increased amount of α-2,3-sialylated N-glycans relative to wild-type soluble CD52 glycoprotein;
(ii) an increased amount of tetra-antennary N-glycans relative to wild-type soluble CD52 glycoprotein; and/or
(iii) an increased amount of di-sialylated O-glycosylation relative to wild-type soluble CD52 glycoprotein.

Optionally, the plurality of soluble CD52 glycoproteins or their fusion proteins of the compositions of the third and alternative third aspects of the invention also have pl's of about 5 to about 6 and/or a decreased amount of N-linked bisecting GlcNAc glycans relative to wild-type soluble CD52 glycoprotein.

The amino acid sequences for soluble CD52 glycoproteins or the CD52 glycoprotein portion of their fusion proteins for the third and alternate third aspects of the invention are optionally as described for the first and alternate first aspects of the invention (or any of their embodiments).

In one embodiment of the third and alternate third aspects of the invention, the N-glycans of the plurality of soluble CD52 glycoproteins or the CD52 glycoprotein portion of their fusion proteins are about 60 to about 70% tri- and tetra-sialylated and/or about 55 to about 65% α-2,3-sialylated.

In another embodiment of the third and alternate third aspects of the invention, the plurality of soluble CD52 glycoproteins or the CD52 glycoprotein portion of their fusion proteins has an increased amount of N-glycans selected from the group consisting of:

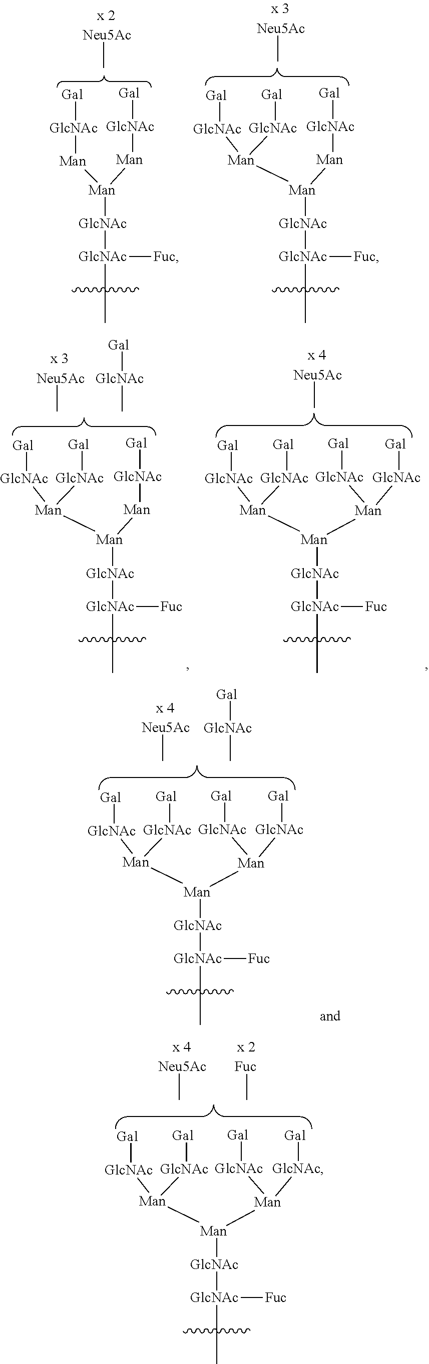

relative to wild-type soluble CD52 glycoprotein.

In an embodiment of the third and alternate third aspects of the invention, the plurality of soluble CD52 glycoproteins or the CD52 glycoprotein portion of their fusion proteins has an increased amount of N-glycans selected from the group consisting of:

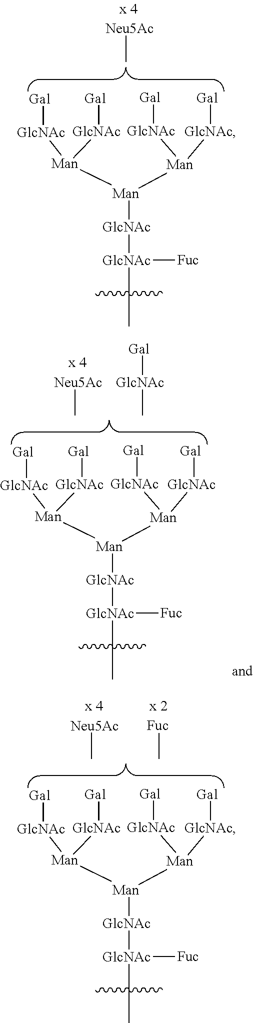

relative to wild-type soluble CD52 glycoprotein.

In an embodiment of the third and alternate third aspects of the invention, the plurality of soluble CD52 glycoproteins or the CD52 glycoprotein portion of their fusion proteins is about 15 to about 20% O-glycosylated.

In another embodiment of the third and alternate third aspects of the invention, the plurality of soluble CD52 glycoproteins or the CD52 glycoprotein portion of their fusion proteins has an increased amount of O-glycans selected from the group consisting of:

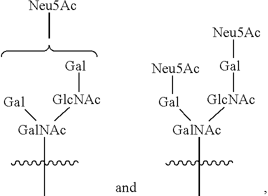

relative to wild-type soluble CD52 glycoprotein.

In an embodiment of the third and alternate third aspects of the invention, the plurality of soluble CD52 glycoproteins or the CD52 glycoprotein portion of their fusion proteins comprise a plurality of CD52 glycoproteins having an amino acid sequence that is at least 60% identical to SEQ ID NO: 3. In this embodiment, the plurality of expressed CD52 glycoproteins or the CD52 glycoprotein portion of their fusion proteins optionally has an increased amount of N-linked glycans linked to the asparagine residue in that peptide (ie Asn3; N3) compared to wild-type CD52 glycoprotein. Alternatively, or in addition, the plurality of CD52 glycoproteins optionally has an increased amount of O-glycosylation at Ser (S) 12 and/or Thr (T) 8 when compared to wild-type CD52.

In some embodiments the compositions of the third embodiment of the invention further comprise insulin, an autoantigen and/or serum.

In a fourth aspect, the present invention provides a CD52 glycoprotein, fusion protein comprising a CD52 glycoprotein or composition according to the first, second or third aspects of the invention or their alternatives (or any of their embodiments), wherein the protein(s) are prepared by expression in a host cell capable of glycosylation. Optionally, the host cell is a mammalian cell, such as a human cell. Optionally, the host cell is Expi 293 or HEK 293.

In an alternative fourth aspect, the present invention provides a CD52 glycoprotein, fusion protein comprising a CD52 glycoprotein or composition according to the first, second or third aspects of the invention or their alternatives (or any of their embodiments), at least some of the α-2,3-sialylation is added enzymatically, optionally by one or more sialyltransferases, such as a sialyltransferase of family GT-42, preferably selected from the group consisting of Cst-II and Cst-I.

The CD52 glycoprotein, fusion protein comprising a CD52 glycoprotein or composition according to the first, second or third aspects of the invention or their alternatives (or any of their embodiments) is optionally isolated or purified. The CD52 glycoprotein, fusion protein comprising a CD52 glycoprotein or composition in the methods or uses of the fifth to eleventh aspects of the invention (or any of their embodiments) is also optionally isolated or purified.

In a fifth aspect, the present invention provides a method of preparing a CD52 glycoprotein, fusion protein comprising a CD52 glycoprotein or plurality of CD52 glycoproteins according to the first, second or third aspects of the invention (or any of their embodiments) comprising:
(i) expressing an amino acid sequence for CD52 glycoprotein, or a CD52 glycoprotein fusion protein, linked to a tag sequence in mammalian cell culture (for example, Expi 293 or HEK 293);
(ii) isolating the CD52 glycoprotein, or a CD52 glycoprotein fusion protein by affinity chromatography using a resin with affinity for the peptide produced from the tag sequence and a suitable eluent.

Optionally, the tag sequence is strep-tag II and the resin is Streptactin resin. Alternative tag sequences will also be known to the skilled person. The eluent is optionally desthiobiotin.

In a sixth aspect of the invention, a fraction of active CD52 glycoproteins and/or their fusion proteins having a pl of about 5 to about 6 is prepared by:
(i) performing anion exchange chromatography on a composition containing proteins according to the third or alternative third aspect of the invention (or any of their embodiments) or a protein prepared according to the fourth or fifth aspect of the invention (or any of their embodiments) to separate the proteins based on their isoelectic point (pl);
(ii) collecting eluted fractions; and
(iii) selecting fractions with a pl of about 5 to about 6.

In a seventh aspect, the present invention provides a method of suppressing effector T-cell function and/or immune response comprising administration to a subject in need thereof:
a CD52 glycoprotein, fusion protein comprising a CD52 glycoprotein or composition according to the first, second or third aspects of the invention (or any of their embodiments); or
a CD52 glycoprotein, fusion protein comprising a CD52 glycoprotein or composition or fraction thereof prepared according to the fourth, fifth or sixth aspects of the invention or their alternatives (or any of their embodiments).

Optionally, the suppressed immune response is an immune response to an autoantigen.

In an eighth aspect, the present invention provides a method of treating or preventing;
a disease or condition mediated by effector T-cell function, inflammation; or
sepsis,
in a subject in need thereof, the method comprising administering a therapeutically effective amount of:
one or more CD52 proteins or fusion proteins thereof or a composition according to the first, second or third aspects of the invention or their alternatives (or any of their embodiments); or
one or more CD52 glycoproteins, fusion proteins comprising a CD52 glycoprotein or compositions or fractions thereof prepared according to the fourth, fifth or sixth aspects of the invention or their alternatives (or any of their embodiments).

In an embodiment, the method comprises mucosal or transdermal administration.

In a ninth aspect, the present invention provides for use of:
a CD52 glycoprotein, fusion protein comprising a CD52 glycoprotein or composition according to the first, second or third aspects of the invention (or any of their embodiments); or
a CD52 glycoprotein, fusion protein comprising a CD52 glycoprotein or composition or fraction thereof prepared according to the fourth, fifth or sixth aspects of the invention or their alternatives (or any of their embodiments);
to suppress effector T-cell function, reduce an immune response, such as an immune response to an autoantigen, or treat or prevent:
a disease or condition mediated by effector T-cell function,
inflammation; or
sepsis.

Optionally, the suppressed immune response is an immune response to an autoantigen.

In a tenth aspect, the present invention provides use of:
a CD52 glycoprotein, fusion protein comprising a CD52 glycoprotein or composition according to the first, second or third aspects of the invention (or any of their embodiments); or
a CD52 glycoprotein, fusion protein comprising a CD52 glycoprotein or composition or fraction thereof prepared according to the fourth, fifth or sixth aspects of the invention or their alternatives (or any of their embodiments)
in the manufacture of a medicament for suppressing effector T-cell function and/or immune response, or the treatment or prevention of:
a disease or condition mediated by effector T-cell function,
inflammation; or
sepsis.

Optionally, the suppressed immune response is an immune response to an autoantigen.

In an eleventh aspect, the present invention provides:
a CD52 glycoprotein, fusion protein comprising a CD52 glycoprotein or composition according to the first, second or third aspects of the invention (or any of their embodiments); or
a CD52 glycoprotein, fusion protein comprising a CD52 glycoprotein or composition or fraction thereof prepared according to the fourth, fifth or sixth aspects of the invention or their alternatives (or any of their embodiments)
for use in therapy.

In an embodiment of the eleventh aspect of the invention, the therapy comprises suppressing effector T-cell function, reducing an immune response, such as an immune response to an autoantigen, or treating or preventing:
a disease or condition mediated by effector T-cell function,
inflammation; or
sepsis.

Optionally, the suppressed immune response is an immune response to an autoantigen.

In the eighth, ninth, tenth and eleventh aspects of the invention, the disease mediated by effector T-cell function is optionally an autoimmune disease, such as type I diabetes or rheumatoid arthritis. Optionally, the condition mediated by effector T-cell function is an allograft rejection or a graft-versus-host reaction.

As used herein, except where the context requires otherwise, the term “comprise” and variations of the term, such as “comprising”, “comprises” and “comprised”, are not intended to exclude further additives, components, integers or steps.

Further aspects of the present invention and further embodiments of the aspects described in the preceding paragraphs will become apparent from the following description, given by way of example and with reference to the accompanying drawings.

### BRIEF DESCRIPTION OF THE DRAWINGS

FIG. 1: Glycosylation analysis of human spleen CD52.

(A) Summed MS profile of released N-glycans from CD52 purified from human spleen tissue. (B) Distribution of O-linked glycans released from human spleen CD52.

FIG. 2: Comparative N-glycoprofiling of recombinant human IgG Fc and CD52.

(A) Proliferation of human PBMCs (3H thymidine uptake) followed 5 days incubation with tetanus toxoid. Histograms show mean±SD of within-assay triplicates, in the presence of different concentration of proteins. The Fc component was cleaved from CD52-Fc with Factor Xa. (B) Summed MS profile of N-glycans released from the Fc (I) and CD52 (II); the latter variant was generated by introducing a point mutation (A297N) into the conventional Fc N-glycosylation site. (C) Factor Xa treated-CD52 was analysed by Western blotting with Campath-H1-HRP antibody.

FIG. 3: Comparison of two recombinant human CD52-Fc variants (I and II) with different immunosuppressive activities.

(A) IFN-g production measured by ELISpot assay from human PBMCs (2×106) in 200 μL/well. Samples were incubated with no antigen or tetanus toxoid in the presence of two different preparations of CD52-Fc (CD52 I or CD52 II; 5, 25, and 50 μg/ml). (B) N-linked glycans released from cleaved CD52 I and CD52 II. The abundance of each N-glycan class is the sum of all EICs measured for all glycans in that class relative to the total of all EICs observed for all N-glycans. (C) EIC of m/z 1140.42-(GlcNAc5Man3Gal2NeuAc1) demonstrating the porous graphitized carbon (PGC) LC-based separation of sialo-glycan isomers observed in CD52 I and CD52 II. (D) Binding of CD52-Fc I and CD52-Fc II (5 and 20 μg/ml) to the α-2,3 sialic acid recognizing lectin MAL-1. (E) ELISpot assay showing activity of CD52-Fc reconstituted with sialic acid in α2-6, α2-3 and/or α2-8 linkages with galactose. The data points in panel (A), (D) and (E) are plotted as mean±SEM of three independent replicate experiments. Data in (B) and (C) are mean±SDs (n=3). Student t test was used to test for significant difference between group means.

FIG. 4: CD52-Fc after fractionation by anion-exchange chromatography.

(A) Anion exchange chromatography on a MonoQ-GL column fractionated the recombinant human CD52-Fc into a gradient of anionic glycoforms displaying a spectrum of pl (FIG. 8). (B) IFN-g ELISpot assay with 2×106 PBMCs in 200 μL/well incubated with no antigen or with anti-CD3/CD28 antibody Dynabeads in the presence of recombinant human CD52-Fc fractions (F29-F53; 5 μg/ml).

FIG. 5: Sialic linkage analysis of active monoQ active fractions.

(A) EICs of the di-sialylated N-glycan m/z 1111.42 of bovine fetuin, known to carry tri-antennary α-2,3-sialylated N-glycans—after sequential α-2,3 sialidase treatment to confirm activity of the α-2,3-specific sialidase. The EICs assess the removal of each of the sialic acid residues. (B) Summed MS of all N-glycans observed for the active CD52 fractions F48 and F49 before and after treatment with α-2,3-specific sialidase. (C) Summary of the immunosuppressive bioactivity, degree of α-2,3 sialylation and bisecting GlcNAcylation of late-eluting MonoQ fractions of particular interest. (D) High-resolution intact mass analysis of the immune suppressive CD52 fractions (F48/F49).

FIG. 6: Mapping the O-glycosylation of recombinant human CD52.

(A) N- and O-glycan occupancy of CD52 I, CD52 II and selected MonoQ fractions (F31 and F46-F51) measured at the protein level after de-N-glycosylation. (B) PGC resolution of O-glycosylated isomers from active fractions m/z 665.22-(GalNAc1 GlcNAc1 Gal2NeuAc2) and m/z 1040.41-(GalNAc1 GlcNAc1 Gal2NeuAc1). (C) EThcD-MS/MS based site localisation analysis showing the peptide backbone fragments and the ions diagnostic of the amino acid site for both aforementioned O-glycans.

FIG. 7: Analysis of CD52 I and CD52 II at the intact peptide level.

(A) The theoretical isotopic distribution of deconvoluted 5871.99 (amu) CD52 glycoform as atri-sialylated (GlcNAc5Man3Gal3NeuAc3Fucose1) or di-sialylated with two outer fucoses (GlcNAc5Man3Gal3NeuAc2Fucose3). The bar graph shows the theoretical isotopic envelopes generated when different amount of these two glycans are present. Experimental isotopic distribution values suggest a population of 90-100% tri-sialylated structures rather than the di-sialylated mass isomer. (B) High-resolution intact mass analysis of CD52 I (pink) and CD52 II (green).

FIG. 8: CD52-Fc fractions resolved in isoelectric focusing (IEF) gel.

Colloidal Coomassie Blue gel showing CD52-Fc fusion protein in MonoQ fractions (F29-54). Fractions showed a gradual decrease in isoelectric point (pl) values.

FIG. 9: High-resolution intact mass analysis of MonoQ fractions (F30 and F47-50).

(A) Intact mass analysis of the inactive MonoQ F30 fraction of the CD52 cleaved from the CD52-Fc fusion protein showing absence of sialic acid molecules. (B) MonoQ fractionation was able to separate CD52 sialylated structures according to their amount of sialic acid as well as number of antennae. Among fractions F47-50, the immune suppressive active F48 and F49 fractions contained large sialylated structures.

FIG. 10: Active MonoQ fractions suppress in a dose-dependent manner.

(A) and (B) IFN-γ production measured by ELISpot assay from human PBMCs (2×105) incubated in IP5 medium with no antigen or anti-CD3/CD28 antibody Dynabeads. (A) Active Mono-Q fractions (F48-49) suppressed in a dose-dependent manner (0.3125, 0.625, 1.25, 2.5, and 5 μg/ml). (B) Adjacent fractions (inactive; F46, F47, F50 and F51) do not suppress despite the increase of protein added (5, 10, 20 and 40 μg/ml). The data points in panels A and B are plotted as mean±SEM of three independent replicates.

### KEY TO THE SEQUENCE LISTING

SEQ ID NO: 1 Human CD52 mRNA transcript (NCBI Reference Sequence: NM_001803.2)
SEQ ID NO: 2 Amino acid sequence of human CD52
SEQ ID NO: 3 12 amino acid soluble peptide of human CD52
SEQ ID NO: 4 Orthologous monkey soluble CD52 peptide
SEQ 10 NO: 5 Orthologous mouse soluble CD52 peptide
SEQ ID NO: 6 Orthologous rat soluble CD52 peptide
SEQ ID NO: 7 Orthologous dog soluble CD52 peptide

### DETAILED DESCRIPTION OF THE EMBODIMENTS

### General Techniques and Definitions

Unless specifically defined otherwise, all technical and scientific terms used herein shall be taken to have the same meaning as commonly understood by one of ordinary skill in the art (e.g., in carbohydrate chemistry, cell culture, molecular genetics, immunology, immunohistochemistry, protein chemistry, proteomics, glycomics and biochemistry).

Unless otherwise indicated, the recombinant protein, cell culture, and immunological techniques utilized in the present invention are standard procedures, well known to those skilled in the art.

The term “and/or”, e.g., “X and/or Y” shall be understood to mean either “X and Y” or “X or Y” and shall be taken to provide explicit support for both meanings or for either meaning.

As used herein, the term “about”, unless stated to the contrary, refers to +/−20%, more preferably +/−10%, more preferably +/−5%, of the designated value. For the avoidance of doubt, the term “about” followed by a designated value is to be interpreted as also encompassing the exact designated value itself (for example, “about 10” also encompasses 10 exactly).

As used herein, except where the context requires otherwise the term “comprise” and variations of the term, such as “comprising”, “comprises” and “comprised”, are not intended to exclude other additives, components, integers or steps.

As used herein, the term “immune response” has its ordinary meaning in the art, and includes both humoral and cellular immunity. An immune response may be mediated by one or more of: T-cell activation, B-cell activation, natural killer cell activation, activation of antigen presenting cells (e.g., B-cells, DCs, monocytes and/or macrophages), cytokine production, chemokine production, specific cell surface marker expression, in particular, expression of co-stimulatory molecules. In a preferred embodiment, the immune response which is suppressed using the methods of the invention is at least effector T-cell function by reducing the survival, activity and/or proliferation of this cell type. In another preferred embodiment, the immune response which is suppressed using the methods of the invention is at least one or more of monocyte, macrophage or dendritic cell function by reducing the survival, activity and/or proliferation of one or more of these cell types. In a further preferred embodiment, the immune response is suppressed to an extent such that it induces tolerance to an antigen such as an autoantigen.

As used herein, the term “tolerance” refers to a state of immune unresponsiveness to a specific antigen or group of antigens to which a subject is normally responsive. Immune tolerance is achieved under conditions that suppress the immune reaction and is not just the absence of an immune response.

As used herein, the terms “treating”, “treat” or “treatment” include administering a therapeutically effective amount of an agent sufficient to reduce or eliminate at least one symptom of disease.

As used herein, the terms “preventing”, “prevent” or “prevention” include administering a therapeutically effective amount of an agent sufficient to prevent the manifestation of at least one symptom of disease.

As used herein, the term “suppressing” includes reducing by any quantifiable amount.

As used herein, the term “subject” refers to an animal, e.g., a mammal. In a preferred embodiment, the subject is mammal, for example a human. Other preferred embodiments include livestock animals such as horses, cattle, sheep and goats, as well as companion animals such as cats and dogs.

As used herein, the term “host” refers to any organism from which soluble CD52 can be isolated or in which soluble CD52 can be produced, by any means. The host may be whole organism or may be a cell derived therefrom. The host may be an animal, e.g., a mammal. In a preferred embodiment, the host is mammalian, for example a human. Other preferred hosts include mice, rats, monkeys, hamsters, guinea-pigs, rabbits, and any animal or cell which may serve as a suitable host from which soluble CD52 can be isolated or in which soluble CD52 can be produced.

As used herein, the term “fusion protein” or variations thereof refers broadly to a protein that is covalently bound or linked to another protein for any period of time.

As the skilled person is aware, the abbreviation “Fuc” refers to fucose, “Man” refers to mannose, “Gal” refers to galactose, “GalNAc” refers to N-acetylgalactosamine, “GlcNAc” refers to N-acetylglucosamine and “Neu5Ac” refers to N-acetylneuraminic acid.

Unless stated otherwise, the terms “soluble CD52 glycoprotein”, “soluble CD52”, “soluble glycoprotein” and variations thereof are used interchangeably herein.

### Soluble CD52 Glycoforms

The present disclosure describes, for the first time, the glycoforms of soluble CD52 glycoprotein fragments that possess bioactivity in suppressing effector T-cell function and/or immune response.

CD52 is a surface glycosylphosphatidylinositol (GPI)-anchored glycoprotein present on most lymphoid cells, initially recognised as the target of complement-binding CAMPATH monoclonal antibodies used therapeutically to deplete lymphocytes. The mRNA transcript of the human CD52 gene is shown in SEQ ID NO: 1 and the translated amino acid sequence is shown in SEQ ID NO: 2.

The sequence for SEQ ID NO:1 is:

| | |
|---|---|
| ctcctggttc aaaagcagct aaaccaaaag aagcctccag acagccctga gatcacctaa | 60 |
| aaagctgcta ccaagacagc cacgaagatc ctaccaaaat gaagcgcttc ctcttcctcc | 120 |
| tactcaccat cagcctcctg gttatggtac agatacaaac tggactctca ggacaaaacg | 180 |
| acaccagcca aaccagcagc ccctcagcat ccagcaacat aagcggaggc attttccttt | 240 |
| tcttcgtggc caatgccata atccacctct tctgcttcag ttgaggtgac acgtctcagc | 300 |
| cttagccctg tgccccctga aacagctgcc accatcactc gcaagagaat cccctccatc | 360 |
| tttgggaggg gttgatgcca gacatcacca ggttgtagaa gttgacaggc agtgccatgg | 420 |
| gggcaacagc caaaataggg gggtaatgat gtaggggcca agcagtgccc agctgggggt | 480 |
| caataaagtt acccttgtac ttgcaaaaaa aaaaaaaaaa aaa | 523 |

The sequence for SEQ ID NO:2 is:

MKRFLFLLLTISLLVMVQIQTGLSGQNDTSQTSSPSASSNISGGIFLF
FVANAIIHLFCFS

Mature CD52 tethered by its GPI anchor comprises only 12 amino acids and an asparagine (N)-linked terminal carbohydrate.

Membrane-anchored CD52 can be cleaved (for example, enzymatically) to release a soluble peptide fragment comprising the amino acid sequence GQNDTSQTSSPS (SEQ ID NO: 3). The soluble CD52 glycoprotein disclosed herein may comprise an amino acid sequence at least 60% identical to the amino acid sequence of SEQ ID NO: 3 or at least 60% identical to the amino acid sequence of other known, orthologous CD52 soluble fragment sequences. Thus, orthologous sequences of the soluble CD52 peptide fragment are encompassed by the present disclosure. Such sequences include but are not limited to the monkey sequence SQNA TSQSSPS (SEQ ID NO: 4), the mouse sequence GQATTAASGTNKNSTSTKKTPLKS (SEQ ID NO: 5), the rat sequence GQNSTAVTTPANKAATTAAATTKAAATTATKTTTA VRKTPGKPPKA (SEQ ID NO: 6), the dog sequence GNSTTPRMTTKKVKSATPA (SEQ ID NO:7), and other orthologous sequences readily identifiable from known CD52 polypeptide and polynucleotide sequences.

It is currently thought that the CD52 peptide functions as a scaffold for presentation of the glycans. Consequently, variation in the sequence is not expected to remove function so long as the amino acids for the required N-glycan and O-glycan links remain.

Percentage identity to any of the amino acid or polynucleotide sequences disclosed herein may be determined by methods known in the art. For example, amino acid and polynucleotide sequences can be compared manually or by using computer based sequence comparison and identification tools that employ algorithms such as BLAST (Basic Local Alignment Search Tool) and others, wherein appropriate parameters for each specific sequence comparison can be selected as would be understood by a person skilled in the art. The amino acid sequence of the peptide portion of the glycoprotein disclosed herein can be at least 65%, at least 70%, at least 75%, at least 80%, at least 85%, at least 90%, at least 95% identical, or at least 99% identical to any one or more of the amino acid sequences identified in SEQ ID NOs: 3, 4, 5, 6 or 7. For example, the amino acid sequence of the peptide portion of the glycoprotein disclosed herein can be 100% identical to any one of the amino acid sequences identified in SEQ ID NOs: 3, 4, 5, 6 or 7.

Isolated soluble CD52 glycoprotein may be used to produce pharmaceutical compositions of the invention. The term “isolated” is used herein to define the isolation of the soluble CD52 glycoprotein so that it is present in a form suitable for application in a pharmaceutical composition. Thus, the glycoprotein disclosed herein is isolated from other components of a host cell or fluid or expression system to the extent that is required for subsequent formulation of the glycoprotein as a pharmaceutical composition. The isolated glycoprotein is therefore provided in a form which is substantially free of other components of a host cell (for example, proteins) which may hinder the pharmaceutical effect of the glycoprotein. Thus, the isolated glycoprotein may be free or substantially free of material with which it is naturally associated such as other glycoproteins, polypeptides or nucleic acids with which it is found in its natural environment, or the environment in which it is prepared (e.g. cell culture) when such preparation is by recombinant DNA technology practised in vitro or in vivo.

Soluble glycoprotein can be isolated from a host cell or fluid or expression system by methods known in the art.

The term “soluble” is used herein to define a peptide or glycoprotein which is not bound to a cell membrane. The soluble peptide or glycoprotein may be able to move freely in any hydrophilic solvent or fluid, such as water, PBS or bodily fluid. For example, the soluble peptide or glycoprotein may be able to circulate in blood.

### Glycoforms of CD52

The CD52 peptide fragment is glycosylated with carbohydrate moieties.

The carbohydrate moieties present on a CD52 glycoprotein can be identified by known methods, such as those described in Schröter et al. (1999) and in Jensen et al. (2012). Such carbohydrate moieties may be identified from CD52 glycoproteins present in any host cell expressing CD52, and particularly lymphocytes, such as CD4+ or CD8+ T-cells, monocytes or genital tract cells, such as sperm cells or epididymal duct cells. Thus, the precise structure of the carbohydrate moiety can be determined by applying methods such as mass spectrometry (e.g. Matrix-assisted Laser Desorption/Ionization-Time of Flight Mass Spectrometry (MALDI-TOF MS), PGC-LC-ESI (Electrospray Ionization)-MS/MS Mass Spectrometry, Mono-Q anion-exchange chromatography, high pH anion exchange chromatography (HPAEC-PAD), methylation analysis, endo-β-galactosidase digestion, and other methods.

The N-glycans may be separated from a CD52 glycoprotein using known cleavage enzymes such as peptide-N4-(N-acetyl-β-D-glucosaminyl)asparagine amidase F (‘PNGase F’ from Flavobacterium meningosepticum, recombinant from Escherichia coli; obtainable from commercial suppliers such as Roche). The N-glycans can be isolated for further characterisation while still attached to the CD52 peptide backbone using known chromatographic methods, such as reversed phase chromatography. The N-glycans may comprise one or more bi-, tri- or tetra-antennary monosaccharide sequence structures, which may be terminally sialylated. For example, the carbohydrate may comprise one or more tetra-antennary monosaccharide sequence structures. The sugars in the sequence may be branched or unbranched to each other. The sugars may comprise a fucose attached to a GlcNAc proximal to the protein Asn. Thus, the carbohydrate may be core fucosylated. The sugars may comprise one or more N-acetyllactosamine (LacNAc; GlcNAc-β1-4 Gal) repeats. Thus, the sugars may comprise polylactosamine units. In addition, the O-glycans may comprise one or more core 1 and 2 structures, which may be terminally sialylated. For example, core structure 2 may contain one or two terminal sialic acids.

Thus, glycans may comprise one or more sialic acids. The one or more sialic acids may be located in any portion of the glycan (usually terminal) and can be α2,3 or α2-6 sialic acids. The CD52 glycoproteins of the invention are preferred to be α2,3 sialylated. The one or more sialic acids may be attached to galactose in β1-4 linkage with N-acetylglucosamine.

### The Bioactivity of Different Glycoforms of Cd52

The present disclosure demonstrates that not all glycoforms of CD52 possess the same level of immunosuppressive bioactivity.

CD52 from human spleen and recombinant forms of human CD52 carry N-glycans that display complex type core fucosylation, abundant sialylation and LacNAc extensions. It was found that there were differences in the specific bioactivity of two recombinant CD52-Fc variants made from different host cells. CD52 in the more bioactive variant of the CD52-Fc fusion protein was abundant in tetra-sialylated N-glycan structures with α-2,3 sialic acid linkage. On the contrary, CD52 in the less bioactive CD52-Fc exhibited a lower abundance of sialylated structures. Using Mono Q anion exchange chromatography, CD52-Fc was separated into a gradient of anionic glycoforms, which exhibited distinctly different immunosuppressive activities. The bioactive glycoforms uniquely displayed an abundance of N-linked tri- and tetra-sialylated glycans (60-70%), high levels of α-2,3 sialylation (58%) and an absence of bisecting GlcNAcylation. Moreover, the most anionic tri- and tetra-sialylated glycans had a unique abundance (15-20%) of core type 2 di-sialylated O-glycans.

Both glycan- and glycopeptide-based analytical approaches were used to correlate CD52 glycosylation with CD52 bioactivity. The glycan approach depended on the high resolving power of PGC columns to separate released glycan isomers and isobaric structures. It was used in conjunction with negative mode ionisation to provide fragment ions of certain glycan structural features (Jensen et al., Harvey et al.). The glycopeptide-based approach allowed analysis of CD52 glycans directly bound to the peptide backbone with the assurance of no interference by the Fc glycans. The two approaches largely corroborated each other adding confidence in the reported structures. Indeed, similar results were found after CD52-Fc glycoform separation by anion exchange chromatography.

Bioactive CD52-Fc was characterized by a high abundance of the α-2,3 sialic acid linkage, removal of the α-2,3 sialic acid linkage removed activity and re-sialylation with α-2,3 sialic acid restored the bioactivity of CD52-Fc.

Regarding CD52 O-glycosylation, the present disclosure characterises for the first time the O-glycans on human spleen CD52. In addition, bioactive recombinant CD52 was found to contain a low abundance (4%) of O-glycans, mainly core type 2 O-glycans with one or two sialic acid residues, on Ser 12 and Thr 8 of SEQ ID 3. Due to the proximity of the N- and O-glycosylation sites of CD52 peptide, it may be that the low degree of O-glycosylation could arise from steric hindrance by the bulky N-glycans.

A dramatic enrichment of O-glycosylation in the bioactive CD52-Fc glycoforms was identified. On SEQ ID:3 at Ser 12, core type 2 di-sialylated O-glycan was observed at 20% compared to 4% in non-active fractions of CD52-Fc. This strongly implies a role for both N- and O-glycosylation in the bioactivity of CD52.

The present disclosure also indicates that fucosylated O-glycans are not required for bioactivity of spleen CD52.

Another striking observation made in the present disclosure was the inverse association between CD52 bioactivity and N-linked bisecting GlcNAcylation.

CD52 in recombinant human CD52-Fc resembled naturally-occurring CD52 purified from human spleen with respect to N- and O-glycosylation, except in the degree of polyLacNAc elongation, which was greater in the native form. Bioactive CD52 was characterized by higher abundance of sialylated structures and polyLacNAcs.

Due to the complex nature of many naturally occurring glycan moieties known to be linked to the extracellular protein portion of human CD52 and the many variations in these structures that may arise from varying glycosylation patterns, it will be understood that the precise nature of the CD52 glycoprotein disclosed herein may vary. As stated above, methods are available to precisely identify particular glycan moieties. In addition, a number of different glycan moieties can be added to the soluble peptide fragment of CD52 by expressing CD52 under varying glycosylation conditions. For example, the soluble glycoprotein disclosed herein may be expressed in and/or isolated from host lymphocyte cells, monocytes or host genital tract cells (e.g. sperm cells, or epididymal duct cells) or seminal fluid and may therefore comprise different carbohydrate groups as a result. It has previously been shown that soluble CD52 present in human semen, similarly to soluble CD52 released from lymphocytes such as Daudi B cells, is capable of suppressing T-cell function and/or an immune response (WO 2013/071355). Alternative host cells providing different glycosylation conditions may be selected for expression of soluble CD52 in order to provide alternative forms of carbohydrate on the soluble glycoprotein.

The glycan may be attached to any one or more amino acid in the peptide which is capable of having a glycan moiety attached thereto. For example, the glycan may be attached to one or more asparagine (N-linked), serine and threonine (O-linked), and also tyrosine, hydroxylysine, hydroxyproline, phosphoserine or tryptophan or other residues if present in the amino acid sequence.

The present disclosure also provides variants, mutants, biologically active fragments, modifications, analogs and/or derivatives of the glycoprotein disclosed herein. Such compounds can be identified by screening for compounds which mimic the structure and/or function of the polypeptide disclosed herein, using methods including any of the methods disclosed herein.

### Soluble CD52 Function

The glycoprotein disclosed herein is preferably capable of suppressing the activity (“function”) of immune cells including lymphocytes (such as aT-cell) and monocytes. For example, the glycoprotein disclosed herein is capable of suppressing one or more of effector T-cell, monocyte, macrophage and dendritic cell function. Effector T-cells, monocytes, macrophages and dendritic cells and their functions will be known to a person skilled in the art.

T-cells can be readily identified by the presence of any of one or more T-cell markers known in the art. The glycoprotein disclosed herein is capable of reducing T-cell proliferation in response to antigen challenge, and/or capable of reducing T-cell cytokine production (such as production of any one or more of IFN-γ, IL-2, IL-10, IL-17, G-CSF, TNF-α, and other cytokines known to be secreted by activated T-cells). For example, soluble CD52 is capable of reducing IFN-γ production by T-cells.

In another example, soluble CD52 is capable of reducing IL-1β secretion by monocytes, macrophages and dendritic cells.

Accordingly, the glycoprotein disclosed herein is capable of reducing an immune response in a host. The inventors have shown that the glycoprotein disclosed herein is capable of reducing effector T-cell function in response to challenge with any antigen. The suppressive function is not dependent on the particular antigen used in the challenge. Thus, the glycoprotein disclosed herein is capable of reducing an immune response to any antigen. In one example, the antigen is an autoantigen.

Any known methods of determining the suppression of T-cell function and/or an immune response can be used, such as (but not limited to) those described in the examples herein. Thus, the methods may comprise determining the effect of the glycoprotein disclosed herein on one or more of effector T-cell, monocyte, macrophage and dendritic cell proliferation and/or on the production of any one or more of IFN-γ, IL-2, IL-10, IL-17, G-CSF, TNF-α, and other cytokines known to be secreted by activated T-cells, monocytes, macrophages or dendritic cells.

### Fusion Proteins

The peptide portion of the CD52 glycoprotein disclosed herein may, for example, be conjugated to a second protein as a fusion protein. The second protein may be any protein capable of increasing the stability and/or solubility of the glycoprotein, of enhancing the process of making the glycoprotein by recombinant methods, or of enhancing the therapeutic effect of the glycoprotein. Thus, the second protein may capable of increasing the half-life of the glycoprotein disclosed herein.

The second protein can be of any suitable length. In one embodiment, the second protein may be relatively short. For example, the second protein may consist of any of 1, 2, 3, 4, 5, 6, 7, 8, 9, 10, or more amino acids. The second protein may comprise at least 1, at least 2, at least 3, at least 4, at least 5, at least 6, at least 7, at least 8, at least 9 or at least 10 amino acids. The second protein may also comprise more than 10 amino acids. For example, the second protein may comprise at least 10, at least 15, at least 20, at least 25, at least 30, or at least 50 amino acids.

In one example, the second protein is an antibody fragment. Suitable antibody fragments include any antibody fragment that is capable of activating the immune system. The antibody fragment may be a fragment crystallizable (Fc) region (which can be a single polypeptide) or any one or more heavy chain constant domains (e.g. CH domains 2, 3 and/or 4) from an Fc region. In one example, the second protein is an Fc fragment. In one example, the second protein is an immunoglobulin G1 (IgG1) Fc fragment.

In another example, the second protein may be a purification tag. Many examples of purification tags are known, and include (without limitation) a His tag, T7 tag, FLAG tag, S-tag, HA tag, c-Myc tag, DHFR, a chitin binding domain, a calmodulin binding domain, a cellulose binding domain, a Strep 2 tag (a purification tag encoding eight amino acids that binds to Strep-Tactin, a specifically engineered streptavidin (Schmidt and Skerra, 2007), and others. In one example, the second protein is a Strep 2 tag.

The second protein may increase the solubility of the expressed protein. Such proteins include (without limitation) NusA, thioredoxin, small ubiquitin-like modifier (SUMO), ubiquitin and others known in the art.

The second protein may increase the solubility of the expressed protein as well as enhancing purification methods. Such proteins include (without limitation) GST, MBP, T7 gene 10, and others known in the art.

The purification tag may optionally be removed from the fusion protein after its production. Suitable methods of removing a purification tag from a fusion protein will vary depending on the particular purification tag used. Such methods will be generally known in the art.

The fusion protein disclosed herein may comprise one or more of any of the second proteins described above, in any combination. Thus, the fusion protein may comprise an antibody fragment (such as an Fc) and a purification tag (such as a Strep 2 tag).

Enzymes that Catalyse the Formation of the Glycosidic Linkage

Enzymes that catalyse the formation of the glycosidic linkage include glycosyl transferases among other options that will be evident to the skilled person.

Glycosyltransferases (GTs) are enzymes that catalyse the formation of the glycosidic linkage to form a glycoside. Glycosyltransferases use nucleotide phosphate sugars as glycosyl donors, and catalyse glycosyl group transfer to a nucleophilic group, usually an alcohol. The product of glycosyl transfer may be an O-, N-, S-, or C-glycoside; the glycoside may be part of a monosaccharide, oligosaccharide, or polysaccharide.

Glycosyltransferases can utilize a range of donor substrates. Sugar mono- or diphosphonucleotides are sometimes termed Leloir donors; the corresponding enzymes are termed Leloir glycosyltransferases.

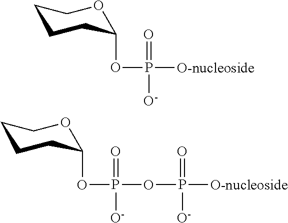

Glycosyltransferases can be classified into families based upon amino acid sequence similarity. As members of a family contain proteins that are related by sequence, they typically share the same mechanism and fold.

The soluble CD52 glycoprotein or fusion protein comprising CD52 glycoprotein disclosed herein may be enriched in α-2,3-sialylation through the action of one or more enzymes. For example, these enzymes may be selected from the group comprising glycosyltransferases, glycosynthases and glycoside hydrolases (when conditioned to operate in the reverse direction). Thus, the CD52 glycoprotein may be enriched in α-2,3-sialylation through the action of a glycosyltransferase (such as GT-42 enzymes Cst-I1 or Cst-1).

### Compositions

The present disclosure provides a pharmaceutical composition comprising any one or more of the soluble CD52 glycoprotein and fusion protein, and a pharmaceutically acceptable excipient.

A pharmaceutically acceptable carrier includes a carrier suitable for use in administration to animals, such as mammals and at least preferably humans. In one example, the term “pharmaceutically acceptable excipient” means excipients approved for use in a pharmaceutical product by a regulatory agency (for example, the US FDA, European EMEA or the Australian TGA) or listed in a pharmacopoeia (such as the U.S. Pharmacopoeia, European Pharmacopoeia or Japanese Pharmacopoeia) or other generally recognized pharmacopeia for use in animals, and more particularly in humans.

The compositions disclosed herein may further comprise an additional therapeutic agent known to suppress effector T-cell function and/or an immune response.

In another embodiment, the composition further comprises an autoantigen. Examples of autoantigens useful in compositions of the invention include, but are not limited to, those listed in Table 1.

TABLE 1
Recombinant or purified autoantigens recognized by autoantibodies
associated with human autoimmune disorders

| Autoantigen | Autoimmune disease |
|---|---|
| A. Cell or organ-specific | |
| autoimmunity | |
| Acetylcholine receptor | Myasthenia gravis |
| Actin | Chronic active hepatitis, primary |
| | biliary cirrhosis |
| Adenine nucleotide translocator | Dilated cardiomyopathy, |
| (ANT) | myocarditis |
| β-Adrenoreceptor | Dilated cardiomyopathy |
| Aromatic L-amino acid | Autoimmune polyendocrine |
| decarboxylase | syndrome type I (APS-I) |
| Asialoglycoprotein receptor | Autoimmune hepatitis |
| Bactericidal/permeability- | Cystic fibrosis vasculitides |
| increasing protein (Bpi) | |
| Calcium-sensing receptor | Acquired hypoparathyroidism |
| Cholesterol side-chain cleavage | APS-I |
| enzyme (CYPIIa) | |
| Collagen type IV α₃-chain | Goodpasture syndrome |
| Cytochrome P450 2D6 (CYP2D6) | Autoimmune hepatitis |
| Desmin | |
| Desmin | Crohn disease, coronary artery |
| | disease |
| Desmoglein 1 | Pemphigus foliaceus |
| Desmoglein 3 | Pemphigus vulgaris |
| F-actin | Autoimmune hepatitis |
| GM gangliosides | Guillain-Barré syndrome |
| Glutamate decarboxylase (GAD65) | Type 1 diabetes, stiff man |
| | syndrome |
| Glutamate receptor (GLUR) | Rasmussen encephalitis |
| H/K ATPase | Autoimmune gastritis |
| 17-α-Hydroxylase (CYP17) | APS-I |
| 21-Hydroxylase (CYP21) | Addison disease |
| IA-2 (ICA512) | Type 1 diabetes |
| Insulin | Type 1 diabetes, insulin |
| | hypoglycemic syndrome (Hirata |
| | disease) |
| Insulin receptor | Type B insulin resistance, acanthosis, |
| | systemic lupus erythematosus (SLE) |
| Intrinsic factor type 1 | Pernicious anemia |
| Leukocyte function-associated | Treatment-resistant Lyme arthritis |
| antigen (LFA-1) | |
| Myelin-associated glycoprotein | Polyneuropathy |
| (MAG) | |
| Myelin basic protein | Multiple sclerosis, demyelinating |
| | diseases |
| Myelin oligodendrocyte | Multiple sclerosis |
| glycoprotein (MOG) | |
| Myosin | Rheumatic fever |
| p-80-Coilin | Atopic dermatitis |
| Pyruvate dehydrogenase complex- | Primary biliary cirrhosis |
| E2 (PDC-E2) | |
| Sodium iodide symporter (NIS) | Graves disease, autoimmune |
| | hypothyroidism |
| SOX-10 | Vitiligo |
| Thyroid and eye muscle shared | Thyroid associated ophthalmopathy |
| protein | |
| Thyroglobulin | Autoimmune thyroiditis |
| Thyroid peroxidase | Autoimmune Hashimoto thyroiditis |
| Thyrotropin receptor | Graves disease |
| Tissue transglutaminase | Coeliac disease |
| Transcription coactivator p75 | Atopic dermatitis |
| Tryptophan hydroxylase | APS-1 |
| Tyrosinase | Vitiligo, metastatic melanoma |
| Tyrosine hydroxylase | APS-I |
| B. Systemic autoimmunity | |
| ACTH | ACTH deficiency |
| Aminoacyl-tRNA histidyl | Myositis, dermatomyositis |
| synthetase | |
| Aminoacyl-tRNA synthetase | Polymyositis, dermatomyositis |
| (several) | |
| Cardiolipin | SLE |
| Carbonic anhydrase II | SLE, Sjögren syndrome, systemic |
| | sclerosis |
| Collagen (multiple types) | Rheumatoid arthritis (RA), SLE, |
| | progressive systemic sclerosis |
| Centromere-associated proteins | Systemic sclerosis |
| DNA-dependent nucleosome- | Dermatomyositis |
| stimulated ATPase | |
| Fibrillarin | Scleroderma |
| Fibronectin | SLE, RA, morphea |
| Glucose-6-phosphate isomerase | RA |
| β32-Glycoprotein I (β32-GPI) | Primary antiphospholipid syndrome |
| Golgin (95, 97, 160, 180) | Sjögren Syndrome, SLE, RA |
| Heat shock protein | Various immune-related disorders |
| Hemidesmosomal protein 180 | Bullous pemphigoid, herpes |
| | gestationis, cicatricial pemphigoid |

### Methods of Treatment

The soluble CD52 glycoprotein and/or fusion protein may be used to suppress effector T-cell function, inflammation or sepsis. Thus, the soluble CD52 glycoproteins, fusion proteins, compositions and fractions described herein, may be used to treat any disease or condition mediated by effector T-cells, inflammatory diseases or disorders and sepsis.

In one example, the disease or condition mediated by effector T-cells may be an autoimmune disease, allograft rejection, a graft-versus-host reaction, or an allergic disease. The term “autoimmune disease” refers to any disease in which the body produces an immunogenic (i.e., immune system) response to some constituent of its own tissue. Autoimmune diseases can be classified into those in which predominantly one organ is affected (eg, hemolytic anemia and anti-immune thyroiditis), and those in which the autoimmune disease process is diffused through many tissues (eg. systemic lupus erythematosus). The autoimmune disease may be (but is not limited to) any one or more of insulin-dependent diabetes mellitus (or type 1 diabetes), insulin autoimmune syndrome, rheumatoid arthritis, psoriatic arthritis, chronic lyme arthritis, lupus, multiple sclerosis, inflammatory bowel disease including Crohn's disease, ulcerative colitis, celiac disease, autoimmune thyroid disease, autoimmune myocarditis, autoimmune hepatitis, pemphigus, anti-tubular basement membrane disease (kidney), familial dilated cardiomyopathy, Goodpasture's syndrome, Sjogren's syndrome, myasthenia gravis, polyendocrine failure, vitiligo, peripheral neuropathy, autoimmnune polyglandular syndrome type I, acute glomerulonephritis, adult-onset idiopathic hypoparathyroidism (AOIH), alopecia totalis, Hashimoto's thyroiditis, Graves' disease, Addison's disease, chronic beryllium syndrome, ankylosing spondylitis, juvenile dermatomyositis, polychondritis, scleroderma, regional enteritis, distal ileitis, granulomatous enteritis, regional ileitis, and terminal ileitis, amyotrophic lateral sclerosis, autoimmune aplastic anemia, autoimmune haemolytic anemia, Behcet's disease, Celiac disease, chronic active hepatitis, CREST syndrome, dermatomyositis, dilated cardiomyopathy, eosinophilia-myalgia syndrome, epidermolisis bullosa acquisita (EBA), giant cell arteritis, Goodpasture's syndrome, Guillain-Barr syndrome, hemochromatosis, Henoch-Schonlein purpura, idiopathic IgA nephropathy, insulin autoimmune syndrome, juvenile rheumatoid arthritis, Lambert-Eaton syndrome, linear IgA dermatosis, myocarditis, narcolepsy, necrotizing vasculitis, neonatal lupus syndrome (NLE), nephrotic syndrome, pemphigoid, pemphigus, polymyositis, primary sclerosing cholangitis, psoriasis, rapidly-progressive glomerulonephritis (RPGN), Reiter's syndrome, stiff-man syndrome, inflammatory bowel disease, osteoarthritis, thyroiditis, and others. In one example, the autoimmune disease is type 1 diabetes. In another example, the autoimmune disease is multiple sclerosis or rheumatoid arthritis. In another example, the condition is an allograft rejection or a graft-versus host reaction. Thus, the methods disclosed herein may comprise administering any one or more of the soluble CD52 glycoproteins, fusion proteins, compositions and/or fractions of the invention to a transplant recipient.

The allergic disease may be (but is not limited to) any one or more of a food allergy, airborne allergy, house dust mite allergy, cat allergy, or bee venom allergy, or other allergy.

Inflammation may arise as a response to an injury or abnormal stimulation caused by a physical, chemical, or biologic agent. An inflammation reaction may include the local reactions and resulting morphologic changes, destruction or removal of injurious material such as an infective organism, and responses that lead to repair and healing.

Inflammation occurs in inflammatory disorders. The term “inflammatory” when used in reference to a disorder refers to a pathological process which is caused by, resulting from, or resulting in inflammation that is inappropriate or which does not resolve in the normal manner. Inflammatory disorders may be systemic or localized to particular tissues or organs. Inflammation is known to occur in many disorders (some of which are autoimmune diseases) which include, but are not limited to, Systemic Inflammatory Response (SIRS); Alzheimer's Disease (and associated conditions and symptoms including: chronic neuroinflammation, glial activation; increased microglia; neuritic plaque formation; Amyotrophic Lateral Sclerosis (ALS), arthritis (and associated conditions and symptoms including, but not limited to: acute joint inflammation, antigen-induced arthritis, arthritis associated with chronic lymphocytic thyroiditis, collagen-induced arthritis, juvenile arthritis, rheumatoid arthritis, osteoarthritis, prognosis and streptococcus-induced arthritis, spondyloarthropathies, and gouty arthritis), asthma (and associated conditions and symptoms, including: bronchial asthma; chronic obstructive airway disease, chronic obstructive pulmonary disease, juvenile asthma and occupational asthma); cardiovascular diseases (and associated conditions and symptoms, including atherosclerosis, autoimmune myocarditis, chronic cardiac hypoxia, congestive heart failure, coronary artery disease, cardiomyopathy and cardiac cell dysfunction, including: aortic smooth muscle cell activation, cardiac cell apoptosis and immunomodulation of cardiac cell function); diabetes (and associated conditions, including autoimmune diabetes, insulin-dependent (Type 1) diabetes, diabetic periodontitis, diabetic retinopathy, and diabetic nephropathy); gastrointestinal inflammations (and related conditions and symptoms, including celiac disease, associated osteopenia, chronic colitis, Crohn's disease, inflammatory bowel disease and ulcerative colitis); gastric ulcers; hepatic inflammations such as viral and other types of hepatitis, cholesterol gallstones and hepatic fibrosis; HIV infection (and associated conditions, including—degenerative responses, neurodegenerative responses, and HIV associated Hodgkin's Disease); Kawasaki's Syndrome (and associated diseases and conditions, including mucocutaneous lymph node syndrome, cervical lymphadenopathy, coronary artery lesions, edema, fever, increased leukocytes, mild anemia, skin peeling, rash, conjunctiva redness, thrombocytosis); nephropathies (and associated diseases and conditions, including diabetic nephropathy, endstage renal disease, acute and chronic glomerulonephritis, acute and chronic interstitial nephritis, lupus nephritis, Goodpasture's syndrome, hemodialysis survival and renal ischemic reperfusion injury); neurodegenerative diseases or neuropathological conditions (and associated diseases and conditions, including acute neurodegeneration, induction of IL-1 in aging and neurodegenerative disease, IL-1 induced plasticity of hypothalamic neurons and chronic stress hyperresponsiveness, myelopathy); ophthalmopathies (and associated diseases and conditions, including diabetic retinopathy, Graves' ophthalmopathy, inflammation associated with corneal injury or infection including corneal ulceration, and uveitis), osteoporosis (and associated diseases and conditions, including alveolar, femoral, radial, vertebral or wrist bone loss or fracture incidence, postmenopausal bone loss, fracture incidence or rate of bone loss); otitis media (adult or paediatric); pancreatitis or pancreatic acinitis; periodontal disease (and associated diseases and conditions, including adult, early onset and diabetic); pulmonary diseases, including chronic lung disease, chronic sinusitis, hyaline membrane disease, hypoxia and pulmonary disease in SIDS; restenosis of coronary or other vascular grafts; rheumatism including rheumatoid arthritis, rheumatic Aschoff bodies, rheumatic diseases and rheumatic myocarditis; thyroiditis including chronic lymphocytic thyroiditis; urinary tract infections including chronic prostatitis, chronic pelvic pain syndrome and urolithiasis; immunological disorders, including autoimmune diseases, such as alopecia aerata, autoimmune myocarditis, Graves' disease, Graves ophthalmopathy, lichen sclerosis, multiple sclerosis, psoriasis, systemic lupus erythematosus, systemic sclerosis, thyroid diseases (e.g. goitre and struma lymphomatosa (Hashimoto's thyroiditis, lymphadenoid goitre); lung injury (acute hemorrhagic lung injury, Goodpasture's syndrome, acute ischemic reperfusion), myocardial dysfunction, caused by occupational and environmental pollutants (e.g. susceptibility to toxic oil syndrome silicosis), radiation trauma, and efficiency of wound healing responses (e.g. burn or thermal wounds, chronic wounds, surgical wounds and spinal cord injuries), septicaemia, acute phase response (e.g. febrile response), general inflammatory response, acute respiratory distress response, acute systemic inflammatory response, wound healing, adhesion, immuno-inflammatory response, neuroendocrine response, fever development and resistance, acute-phase response, stress response, disease susceptibility, repetitive motion stress, tennis elbow, and pain management and response.

The invention will now be described with reference to the following, non-limiting examples.

### EXAMPLES

Experimental Procedures

### Human Blood and Spleen Donors

Cells were isolated from human blood buffy coats (Australian Red Cross Blood Service, Melbourne, VIC, Australia) or blood of de-identified healthy volunteers with informed consent through the Volunteer Blood Donor Registry of The Walter and Eliza Hall Institute of Medical Research (WEHI), following approval by WEHI and Melbourne Health Human Ethics Committees. Peripheral blood mononuclear cells (PBMCs) were isolated on Ficoll/Hypaque (Amersham Pharmacia, Uppsala, Sweden), washed in phosphate-buffered saline (PBS) and re-suspended in IMDM medium containing 5% pooled, heat-inactivated human serum (PHS; Australian Red Cross, Melbourne, Australia), 100 mM non-essential amino acids, 2 mM glutamine and 50 μM 2-mercaptoethanol (IP5 medium).

Healthy human spleen from cadaveric organ donors were obtained from Australian Islet Transplant Consortium and trained coordinators of Donate Life from heart-beating, brain dead donors with informed written consent of next of kin. All studies were approved by WEHI Human Research Ethics Committee (Project 05/12).

Purification of Native CD52 from Human Spleen

Frozen human spleen tissue (10 mg) was homogenized with three volumes of water. Homogenate was mixed with methanol and chloroform 11:5.4 volumes, respectively. Samples were left to stir for 30 min and allowed to stand for one hour. The upper (aqueous) phase was collected, evaporated, dialyzed and freeze dried. NHS-activated Sepharose 4 Fast Flow resin was incubated with 1 mg of purified anti-CD52 antibody in 0.5 mL of PBS for 3 h at RT. The mixture was incubated overnight at 4° C. and quenched with 1 M ethanolamine. A Bio-Rad 10-mL Poly-Prep column was used for packing and resins were washed with sequential treatment of 5 mL of PBS, 5 mL of pH 11.5 diethylamine, and 5 mL of PBS/0.02% sodium azide. The column was stored at 4° C. in 5 mL of PBS/0.02% sodium azide before use. Spleen extracts were solubilized with 2 mL of 2% sodium deoxycholate in PBS, and then added to the packed column and washed with 5 mL of PBS containing 0.5% sodium deoxycholate. The sample was eluted with six times 500 μl of elution buffer (50 mM diethylamine, 500 mM NaCl, pH 11.5) containing 0.5% sodium deoxycholate. The eluate was collected, neutralized with 50 μl of HCl (0.1M) and dialyzed against PBS and water.

### CD52 Recombinant Proteins

Human CD52-Fc recombinant proteins; CD52-Fc I (Expi293), CD5-Fc II (HEK293) and CD52-Fc III (Expi293) were produced as described (Bandala-Sanchez et al. (2013)). The signal peptide sequences joined to human IgG1 Fc were constructed with polymerase chain reaction (PCR) then digested and ligated into a FTGW lentivirus vector or pCAGGS vector for the transfection of FreeStyle HEK293F and Expi293 cells. The construct included a flexible GGSGG linker, a strep-tag II sequence for purification (Schmidt et al.), and a cleavage sites for Factor Xa protease between the signal peptide and Fc molecule. The recombinant proteins were purified from the medium by affinity chromatography on Streptactin resin and eluted with 2.5 mM desthiobiotin (Bandala-Sanchez et al. (2013)).

### ³H-Thymidine Incorporation Assay

PBMCs (2×10⁵ cells/well) in IP5 medium were incubated for up to 3 d at 37° C. in 5% (X/X) CO2 in 96-well round-bottomed plates with or without tetanus toxoid (10 Lyons flocculating units per ml), and various concentrations of CD52-Fc or control Fc protein, in a total volume of 200 μL. In some wells, ³H-thymidine (1 μCi) was added and, after 16 h, cells were collected and radioactivity in DNA measured by scintillation counting.

### ELISpot Assay

PBMCs (2×105 cells/well) were cultured in 200 μL of IP5 medium in triplicate wells of a 96-well ELISpot plate (PVDF MultiScreen) from Merck Millipore (Bayswater, Australia) containing anti-IFN-γ monoclonal antibody pre-bound (1 μg/mL) at 4° C. PBMCs were incubated with tetanus toxoid (10 Lfu/mL) added to the wells together with CD52-Fc I, CD5-Fc II and CD52-Fc III (5, 25 and 50 μg/mL). After 24 h, cells were removed by washing and IFN-γ spots were developed by incubation with biotinylated anti-IFN-γ antibody (1 μg/mL) followed by streptavidin-alkaline phosphatase and BCIP/NBT colour reagent (Resolving Images, Melbourne, Australia).

### Lectin ELISA

A 96-well flat-bottom plate was coated with 20 μg/mL of Maackia amurensis lectin (MAL-1) overnight at 4° C. and subsequently blocked with 200 μl of 1% BSA for 1 h. After washing with PBS, CD52-Fc (20 μg/mL) was added and incubated at RT for 1 h and washed twice with PBS. After washing with PBS, 50 μl of a 1:1000 dilution of HRP-conjugated antibody to CD52 (Campath H1; 1 μg/mL) was added and incubated at RT for 1 h. 50 μl of TMB substrate was added and colour development stopped by addition of 50 μl of 0.5 M H2SO4. Absorbance was measured at 450 nm in a Multiskan Ascent 354 microplate photometer (Thermo Labsystems, San Francisco, Calif.).

### De-Sialylation and Re-Sialylation of Recombinant CD52-Fc Protein

De-sialylation and re-sialylation of recombinant CD52-Fc proteins were performed by a modification of the method of Paulson and Rogers. Briefly, CD52-Fc (500 μg/each) was incubated with Clostridium perfringens type V sialidase (50 mU/mL) for 3 h at 37° C. to remove all types of sialic acids. Samples were then passed through a Protein G-Sepharose column, which was washed twice with PBS before the bound protein was eluted with 0.1 M glycine-HCl, pH 2.8 into 1 M Tris-HCl, pH 8.0, followed by dialysis against PBS. Binding to MAL-I lectin was performed to confirm removal of sialic acids. CD52-Fc from Expi293 cells was then incubated with either of two sialyltransferases, PdST6Gall which restores sialic acid residues in α-2,6 linkage with underlying galactose or Cstll which restores sialic acid residues in α-2,3 linkage with galactose, in the presence of 0.46 mM-0.90 mM CMP-N-acetylneuraminic acid sodium salt (Carbosynth, Compton Berkshire, United Kingdom) for 3 h at 37° C. The different CD52-Fc (III) proteins with different linkages (α-2,3 or α-2,6) were passed through Protein G-Sepharose columns, washed twice with PBS and eluted with 0.1 M glycine-HCl, pH 2.8, into 1 M Tris-HCl, pH 8.0, followed by dialysis against PBS. Samples were freeze-dried, re-suspended in PBS at 200 μg/mL and stored at −20° C.

### Fc Fragment Removal

CD52-Fc recombinant protein fractions (50-200 μg) were incubated with 4 μL of Factor Xa protease (purified from bovine plasma, New England Biolabs, United States) in a total volume of 1 mL of cleavage buffer (20 mM Tris-Hcl, pH 8, 100 mM NaCl, 2 mM CaCl₂)). Samples were incubated overnight at RT. Samples were mixed three times with Protein G-Sepharose beads for 1 h at RT and centrifuged at 10,000 rpm for 15 min. Fc fragment removal was confirmed by Western blot using anti-human IgG (Fc specific produced in goat; Sigma, United States) and anti-CD52 (rabbit) antibodies (Santa Cruz Biotechnology, United States).

### N- and O-Linked Glycan Release for Mass Spectrometry Analysis

Mono Q fractionated and whole (non-fractionated) recombinant CD52-Fc were dot-blotted on a PVDF membrane prior to N-glycan release by an overnight incubation with 2.5 U N-glycosidase F (PNGase F, Elizabethkingia miricola, Roche) at 37° C. followed by a NaBH₄ reduction (1 M NaBH₄, 50 mM KOH) for 3 h at 50° C. The O--glycans were subsequently released by overnight reductive β-elimination using 0.5 M NaBH₄, 50 mM KOH at 50° C. The released and reduced N- and O-glycans were thoroughly desalted prior to the LC-MS/MS as described previously (Jensen et al.). Soluble CD52 with the Fc removed does not adhere to PVDF so was kept in-solution prior to N- and O-glycan release.

### Mass Spectrometry and Data Analysis of Released Glycans

The separation of glycans was performed by using a porous graphitised carbon (PGC) liquid chromatography (LC) column (5 μm particle size, 180 μm internal diameter×10 cm column length; Hypercarb KAPPA Capillary Column, Thermo Scientific) operated at a constant flow rate of 4 μl/min using a Dionex Ultimate 3000 LC (Thermo Scientific). The separated glycans were detected online using liquid chromatography-electrospray ionisation tandem mass spectrometry (LC-ESI-MS/MS) using an LTQ Velos Pro mass spectrometer (Thermo Scientific). The PGC column was equilibrated with 10 mM ammonium bicarbonate (Sigma Aldrich) and samples were separated on a 0-70% (v/v) acetonitrile in 10 mM ammonium bicarbonate gradient over 75 min. The ESI capillary voltage was set at 3.2 kV. The full auto gain control was set to 80,000. MS1 full scans were made between m/z 600-2000. All glycan mass spectra were acquired in negative ion mode. The LTQ mass spectrometer was calibrated with a tune mix (Pierce™ ESI negative ions, Thermo Scientific) for mass accuracy of 0.2 Da. The CID-MS/MS was carried out on the five most abundant precursor ions in each full scan by using 35 normalized collision energy. Possible monosaccharide compositions were provided by GlycoMod (Expasy, http://web.expasy.org/glycomod/) based on the molecular mass of glycan precursor ions (Cooper et al.). Analysis of MS/MS spectra was performed with Thermo Xcalibur Qual browser software. Possible glycan structures were identified based on diagnostic fragment ions 368 for core fucosylation and others as reported (Everest-Dass et al.), and BY- and C/Z-glycan fragments in the CID-MS/MS spectra. A mass tolerance of 0.2 Da was allowed for both the precursor and product ions. The relative abundances of the identified glycans were determined as a percentage of the total peak area from the MS signal strength using area under the curve (AUC) of extracted ion chromatograms of glycan precursor ion (Harvey et al.).

### Profiling the N- and O-Glycans on the CD52 Peptide

MonoQ fractionated and unfractionated CD52 glycoforms without the Fc were desalted on C18 micro-SPE stage tips (Merck-Millipore). Elution was performed with 90% (v/v) Acetonitrile (ACN) and samples were dried and redissolved in 0.1% (v/v) Formic acid (FA). The desalted CD52 glycopeptides were analysed by ESI-LC-MS in positive ion polarity mode using a Quadrupole-Time-of-flight (Q-TOF) 6538 mass spectrometer (Agilent technologies)-HPLC (Agilent 1260 infinity). In parallel experiments, N-glycosidase F was used to remove N-glycans from some samples of CD52 (with a resulting Asn->Asp conversion i.e. +1 Da) to enable better ionization of the highly heterogeneous and anionic CD52 glycopeptides. The N- and O-glycan occupancy was determined by comparing the AUC of the deamidated and O-glycosylated CD52 glycoforms. Samples (roughly 500 ng) were injected onto C8 column (Protecol C8, 3 μm particle size, 300 A pore size, 300 nm inner diameter 10 cm length, SGE analytical science). The HPLC gradient was made starting with 0.1% (v/v) FA with a linear rise to 60% (v/v) ACN 0.1% (v/v) FA over 30 min. The column was then washed with 99% ACN (v/v) for 10 min before re-equilibration with 0.1% FA for another 10 min. The flow rate was set to 4 μL/min with an optimised fragmentor positive potential of 200 V with the following MS setting: m/z range 400-2500, nitrogen drying gas flow rate 8 L/min at 300° C., nebulizer pressure was 10 psi, capillary positive potential was 4.3 kV, skimmer potential was 65 V. The mass spectrometer was calibrated with a tune mix (Agilent technologies) to reach a mass accuracy typically better than 0.2 ppm. MassHunter workstation vB.06 (Agilent technologies) was used for analysis and deconvolution of the resulting spectra. The previously determined glycans from the PGC-LC-MS/MS analysis were used to guide the assignment of glycoforms to deconvoluted CD52 peptides based on the accurate molecular mass.

### Mono Q Column Fractionation

CD52-Fc III was diluted into 5 mL 50 mM Tris-HCl, pH 8.3 and applied to a Mono Q column (Mono Q 5/50 GL, GE Life Sciences). The column was washed with 10 column volumes of 50 mM Tris-HCl, pH 8.3 and then eluted with 50 column volumes of 50 mM Tris-HCl, 500 mM NaCl, pH 8.3 in 0.5 mL fractions. Fractions were then collected and analyzed by isoelectric focusing (IEF).

### IEF

Novex pH 3-10 IEF gels were used for pl determination. CD52-Fc fractions were loaded with sample buffer and run at 100 V for 2 h, then at 250 V for 1 h and, finally, the voltage was increased to 500 V for 30 min. After electrophoresis, the gel was carefully transferred to a clean container, washed and fixed with 20% trichloroacetic acid (TCA) for 1 h at RT, rinsed with distilled water, stained with colloidal Coomasie blue for 2 h at RT, and thoroughly destained with distilled water.

### Sequential Sialidase Treatment

N-glycans released from Fc cleaved CD52 (roughly 2 μg) were treated with α-2-3-specific sialidase (1 mU, Merck) and broad (α-2-3,6,8 sialidase-reactive) sialidase V. cholera (1 mU, Sigma Aldrich). Both reactions were carried out in 50 mM sodium phosphate reaction buffer at 37° C. for 3 h. Desialylated CD52 N-glycans were dried and solubilised in water for downstream MS analysis. Fetuin was used as positive control for successful sialic acid removal since, like cleaved CD52, this model glycoprotein carries multi-antennary sialylated N-glycans.

### EThcD Fragmentation for O-Glycan Site Localisation on the CD52 Peptide

Fractionated CD52 glycoforms were treated with PNGase F prior to 0-glycan site localisation analysis. CD52 peptides were analysed using a Dionex 3500RS nanoUHPLC coupled to an Orbitrap Fusion™ Tribrid™ Mass Spectrometer in positive mode with the same LC gradient mentioned in ‘Profiling the N- and O-glycans on intact CD52’,-but with a nano-flow (250 nL/min). The following MS settings were used: spray voltage 2.3 kV, 120k orbitrap resolution, scan range m/z 550-1,500, AGC target 400,000 with one microscan. The HCD-MS/MS used 40% nCE. Precursors that resulted in fragment spectra containing diagnostic oxonium ions for glycopeptides i.e. m/z 204.08671, 138.05451 and 366.13961, were selected for a second EThcD (nCE 15%) fragmentation. The analysis of all fragment spectra was carried out using Thermo Xcalibur Qual browser software with the aid of Byonic (v2.16.11, Protein Metrics Inc) using the following parameters: precursor mass tolerance 6 ppm, fragment mass tolerance 1 Da and 10 ppm to respectively account for possible proton transfer during ETD fragment formation and the MS/MS resolution, deamidated (variable) and two core type 2 O-glycans, previously seen in intact mass analysis.

Data are expressed as mean±standard deviation (SD). The significance of differences between groups was determined by t-test, with Prism software (GraphPad Software). p<0.05 was used throughout as the significance threshold.

### Example 1: Analysis of N- and O-Glycosylation of Human Spleen-Derived CD52

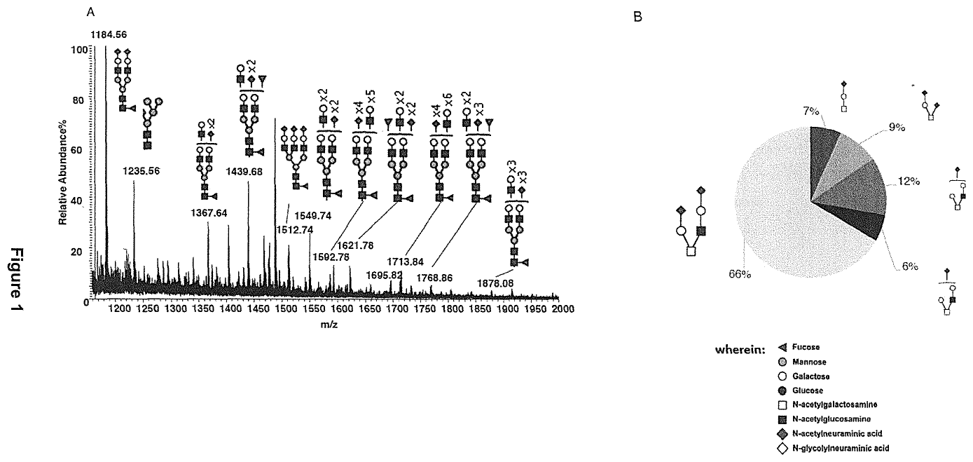

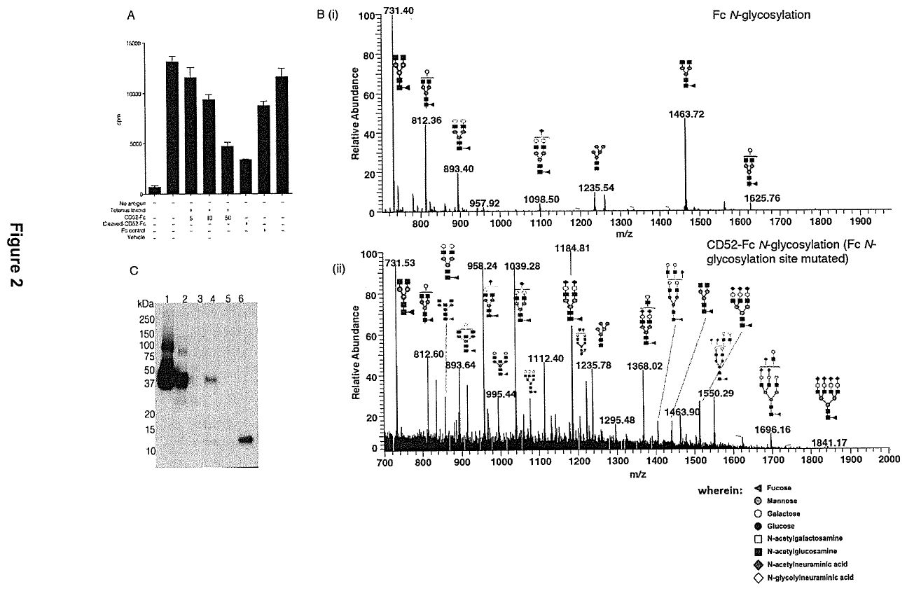

To characterize the natural glycosylation of human CD52, CD52 from human spleen was purified. A comprehensive analysis of released N- and O-glycans by porous-graphitised carbon was performed (PGC)-ESI-MS/MS (FIG. 1A, B). High N-glycosylation heterogeneity was confirmed, expressed as multi-antennary sialylated N-glycans with abundant polyLacNAc extensions (FIG. 1A). Similar N-glycans have been previously reported for naturally occurring human CD52 (Treumann et al.). The O-glycosylation profile was characterized as core type 1 and core type 2 sialylated structures with mainly (66%) di-sialylated core type 2 O-glycans (FIG. 1B). This glycan heterogeneity suggests that particular bioactive glycoforms of CD52 exist. Further experiments were performed to determine whether such heterogeneity is reflected in the recombinant form of human CD52 (see Examples 2-7).

### Example 2: Assessing the Impact of N-Glycosylation on Recombinant CD52 Produced from Different Host Cells with Fc Carrier Protein

Human CD52 was engineered as a recombinant fusion protein conjugated with an IgG1 Fc fragment as described (Bandala-Sanchez et al. (2013)). The two recombinant human CD52-Fc batches generated for this study recapitulated the previously observed immuno-suppressive bioactivity (FIG. 2A). However, the Fc has a single N-linked glycosylated site at N297 (FIG. 2B (i)), which had to be considered in characterizing and assessing the impact of the N-glycosylation of recombinant CD52-Fc. This was addressed in two ways: i) by analysing a recombinant form of human CD52-Fc in which Fc contained an N297A mutation, allowing analysis of CD52 N-glycosylation profile at the released glycan level without interference from the Fc N-glycan (FIG. 2B (ii)), and 2) by removal of the Fc component from CD52-Fc by Factor Xa proteolysis of a cleavage site appropriately incorporated in the CD52-Fc construct, as shown by a Western blot using a specific antibody for CD52 (FIG. 2C).

### Example 3: Assessing the Bioactivity of Recombinant CD52 Glycoforms

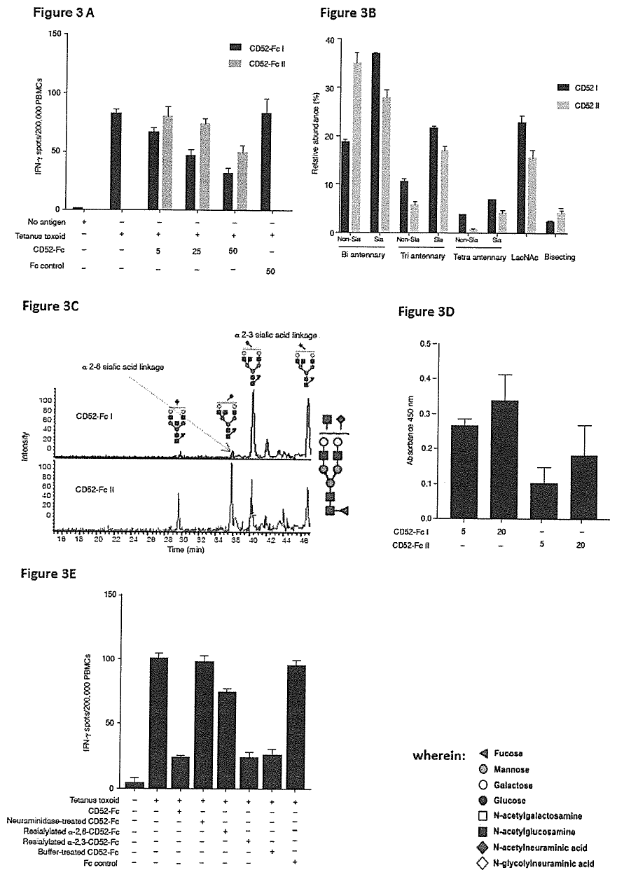

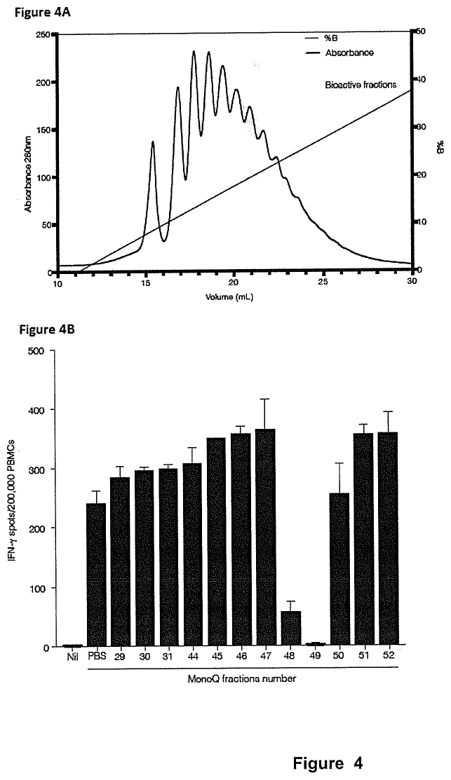

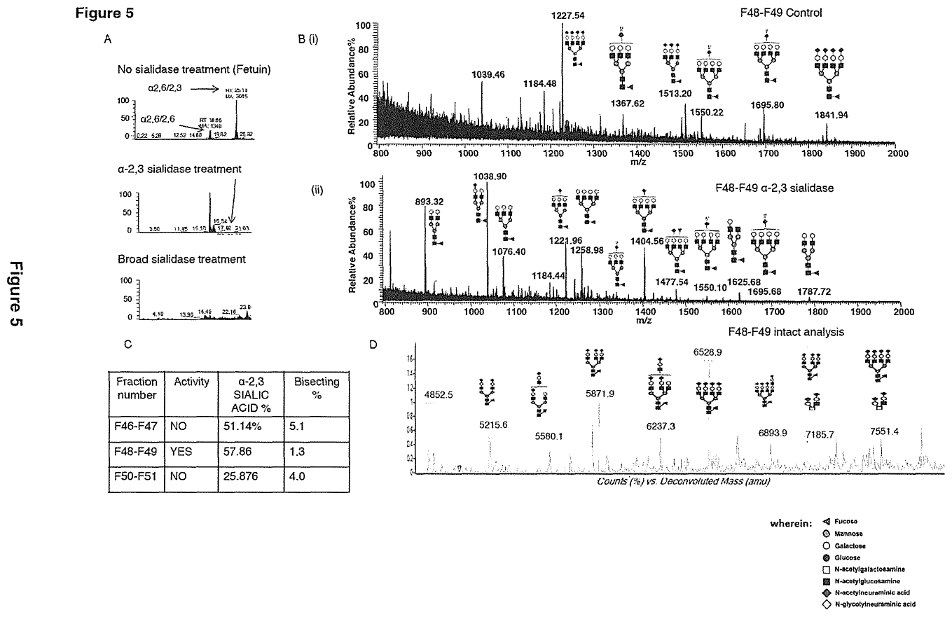

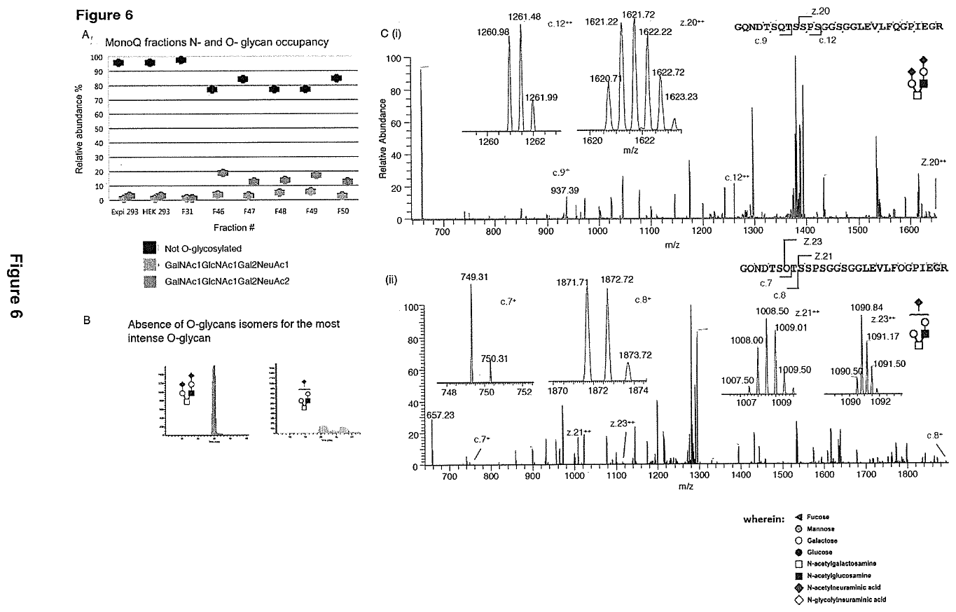

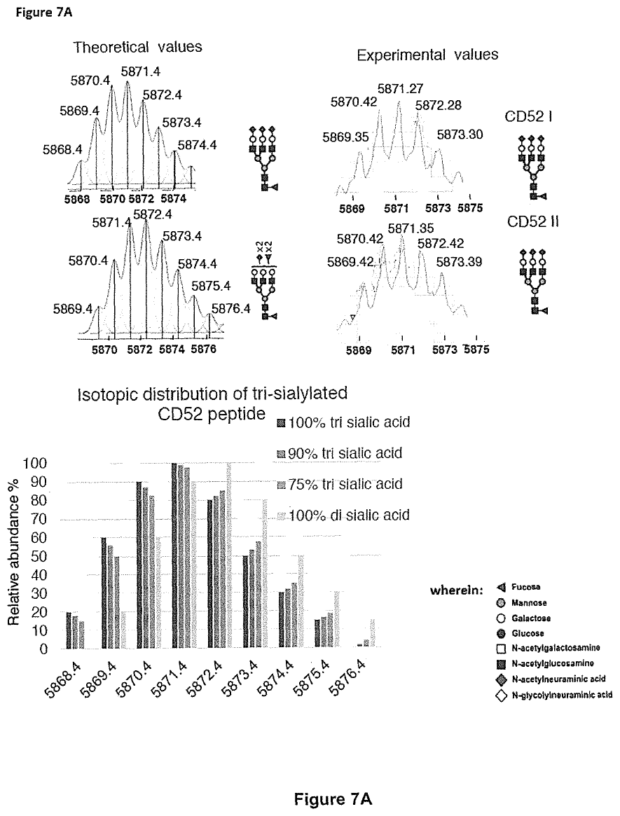

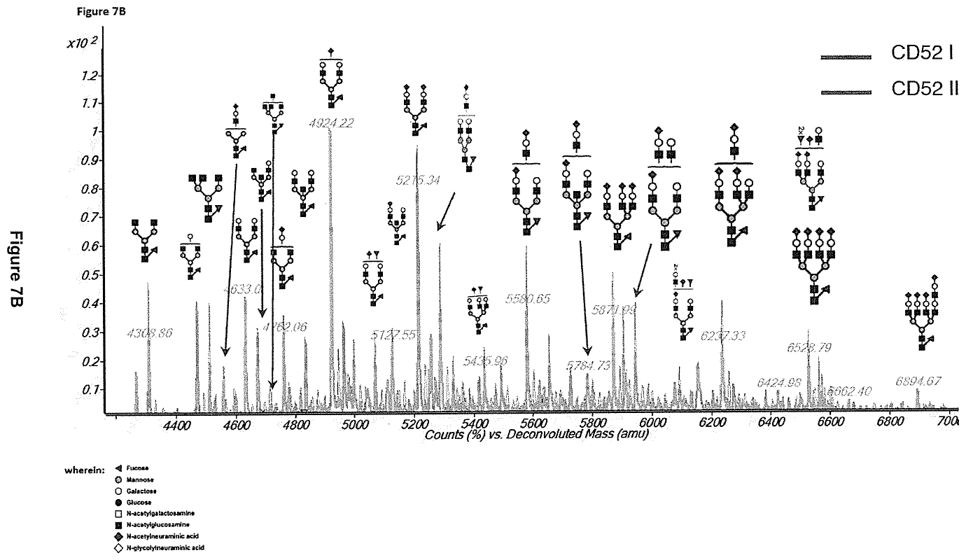

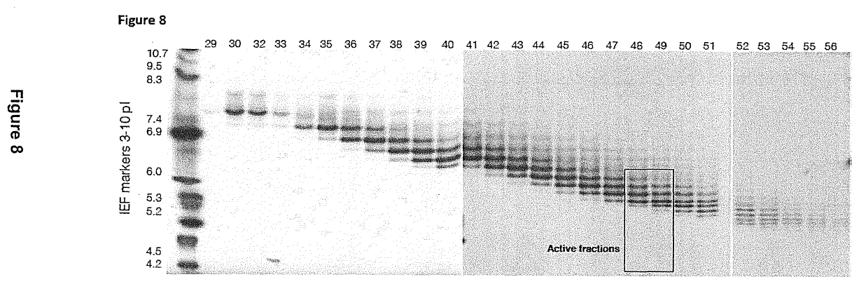

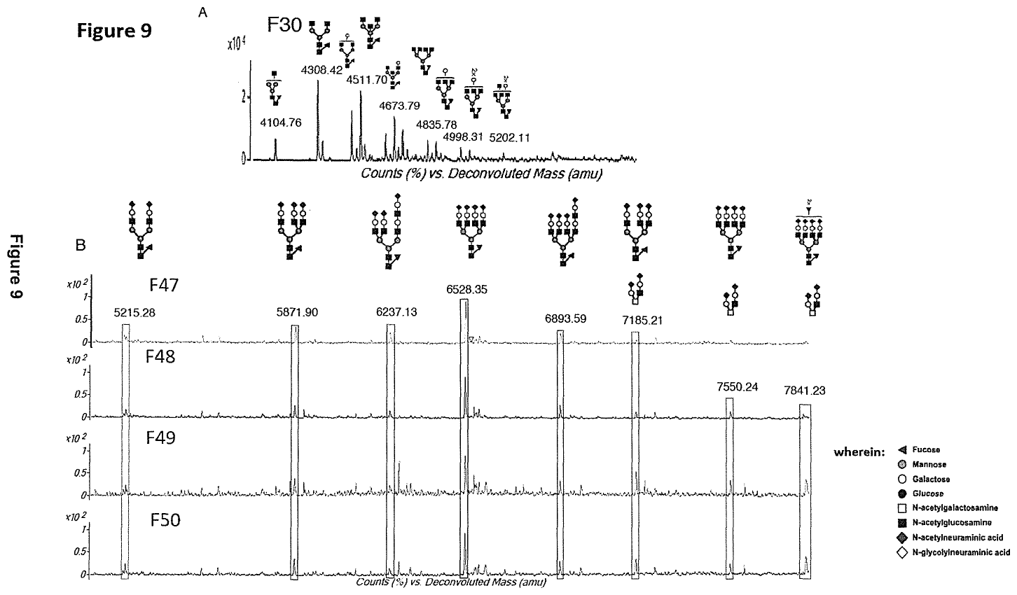

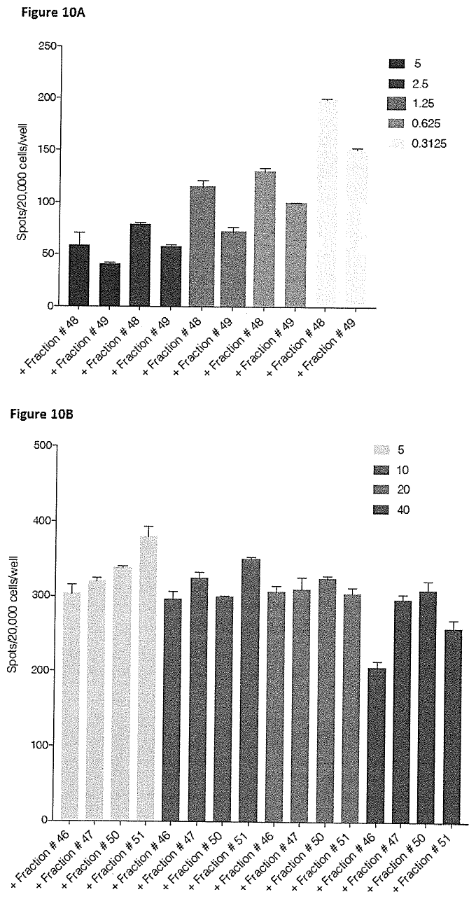

It was noted that the specific bioactivity of recombinant CD52-Fc varied from batch to batch. Therefore, the impact of sialylation between two CD52-Fc variants made from different host cells that were shown display higher and lower immunosuppressive activity was compared, here referred to respectively as CD52-Fc I (from Expi 293 cells) and CD52-Fc II (from HEK 293 cells) (FIG. 3A).

N-glycans were released via in-solution treatment with PNGase F and subsequently analysed by PGC-ESI-MS/MS (Jensen et al.). N-glycans on cleaved CD52 I had greater relative abundances of bi-, tri- and tetra-antennary sialylated glycans compared to CD52 II (FIG. 3B). Also, CD52 I displayed a significantly higher relative abundance of sialylated structures possibly containing LacNAc moieties (FIG. 3B). Not only the numbers of antennae, but also their degree of sialylation differed between the two recombinant CD52 glycoforms: tetra-sialylated N-glycans were significantly more abundant in CD52 I (6.9±0.9%) compared to CD52 II (4.9±1.3) (p<0.05). In contrast, CD52 II displayed significantly greater abundance of non-sialylated bi-antennary and bisecting structures (30% and 5% compared to 19% and 2%, respectively) (FIG. 3B).

After the removal of Fc, recombinant CD52 I and CD52 II were then subjected to high-resolution intact peptide analysis using C8-LC-ESI-MS. Both proteins showed N-glycosylation profiles similar to those of released glycans. The high resolution of the Q-TOF instrumentation used even in the high m/z range enabled the identification of very elongated sialylated antennary structures including searching for N-glycans carrying Lewis-type structures (antenna-type fucosylation). The experimental isotopic distribution of both variants of recombinant CD52 matched the theoretical isotopic distribution of the 90% tri-sialylated (non-Lewis fucosylated) CD52 glycoforms, which indicate that the main glycoforms of recombinant CD52 do not carry Lewis-type fucosylation (FIG. 7A). The more bioactive CD52 I displayed a higher level of multi-antennary sialylated and possible LacNAc elongated structures (FIG. 7B).

### Example 4: Assessing the Importance of α-2,3 Sialylated N-Glycans for CD52 Glycoforms

CD52 N-glycans displaying α-2,3 sialylation preferentially bind to Siglec-10. PGC-LC-MS/MS glycan analysis and Maackia amurensis-1 (MAL-1) lectin blotting were used to identify any differences in sialic acid linkage between the two variants of recombinant CD52-Fc (CD52-Fc I and CD52-Fc II). MAL-I has a known preferential recognition of glycoconjugates displaying α-2,3 sialylation. Despite the high separation power of PGC for sialoglycans, this technique has difficulty resolving very large multi-antennary sialylated glycans, but can easily discriminate between α-2,3 and α-2,6-sialylation on the more common bi- and tri-antennary N-glycans. Several abundant bi-antennary α-2,3 sialoglycans were observed on CD52 I. For one sialylated glycan, m/z 1140.42-(GlcNAc5Man3Gal2NeuAc1), only the α-2,3 sialic acid glycan isomer was observed on CD52 I. On the other hand, the less bioactive CD52 II carried both α-2,3 and α-2,6 sialo-N-glycans (FIG. 3C). This differential sialyl linkage presentation between the two recombinant CD52 variants was supported by MAL-I lectin binding, which was higher for the more bioactive CD52-Fc I (FIG. 3D). MAL-I lectin favours binding to α-2,3 sialic acid linked tri- and tetra-sialylated N-glycans (Wang and Cummings). The importance of the α-2,3 sialylation for bioactivity of CD52-Fc was confirmed in a parallel experiment where the immuno-suppressive activity of sialidase-treated and re-sialylated CD52-Fc was determined relative to the original recombinant variant. Treatment of CD52-Fc with sialidase completely abolished its immunosuppressive activity, which was fully restored upon re-sialylation with α-2,3, but not α-2,6 sialylation (FIG. 3E). Overall, these findings indicate that the bioactivity of CD52-Fc is associated with the presence of α-2,3-linked tetra-sialylated N-glycans found on CD52 and not the attached Fc.

### Example 5: Resolution of Active CD52 Glycoforms by Anion Exchange Chromatography

Anion exchange chromatography was performed on a MonoQ column in order to separate recombinant CD52-Fc variants based on their degree of sialylation, with the aim of identifying the most bioactive forms (FIG. 4A). The increasing degree of sialylation (decreasing isoelectric point [pl]) of CD52-Fc in the collected fractions was confirmed by isoelectric focusing (IEF) (FIG. 8) and mass spectrometry. The released N-glycans from fractions 46 to 51 (F46-F51) revealed a gradual increase in sialic acid content, and structures containing a higher number of antennae (Table 2), as shown also from intact glycopeptide analysis (FIG. 9). Released and intact glycan analysis from fraction 30 revealed various GlcNAc and Gal capped structures and a complete absence of sialic acid moieties (Table 2 and FIG. 9). Remarkably, only two fractions, F48 and F49, with pls in the 5-6 range displayed significant immunosuppressive activity (FIG. 4B). The adjacent fractions were not bioactive, even at higher concentrations of protein eg 20 and 40 μg/ml (FIG. 10). These late-eluting, uniquely bioactive fractions (F48-49) were highly enriched (60-70%) in tri- and tetra-sialylated glycans, further confirming that sialylation strongly impacts the bioactivity of CD52.

### Example 6: Assessment of α-2,3 Sialylation in Active CD52 MonoQ Fractions

It is challenging to determine the sialylation linkages of large, multi-sialylated N-glycans by mass spectrometry. Therefore, differences in sialic acid linkage of active and adjacent non-active MonoQ fractions were probed by α-2,3-specific sialidase treatment. The linkage-specific activity of α-2,3 sialidase was confirmed on bovine fetuin as a control protein, which demonstrated specific removal of α-2,3-linked sialic acid residues from the known bi-antennary sialylated glycan m/z 1111.52-(FIG. 5A). The glycan products resulting from α-2,3 sialidase treatment of the active fractions of CD52 were determined via PGC-LC-MS/MS (FIG. 5B (i) and (ii)). The active MonoQ fractions (F48/F49) had a higher proportion of α-2,3 sialic acid (58%) compared to adjacent earlier (F46, F47) and later (F50, F51) eluting fractions (51% and 25%, respectively) and less bisecting structures than the adjacent non-active fractions (1%, compared to 4% and 5%, respectively) (FIG. 5C). Finally, the profile of the most active CD52 fractions at the intact peptide level supported a predominance of tri- and tetra-antennary sialylated structures (FIG. 5D).

### Example 7: Assessment of O-Glycans of CD52

Initially, O-glycosylation analysis of de-N-glycosylated CD52 at the intact peptide level revealed that both variants of recombinant CD52 (CD52 I and CD52 II) had very low (4%) 0-glycan occupancy (FIG. 6A), casting doubt on the relevance of O-glycosylation for CD52 activity. Non-deamidated signatures were absent in the spectra for both CD52 I and II, indicating that the CD52 peptides were fully N-glycosylated (black symbols, FIG. 6A). Like human spleen CD52, the recombinant CD52 proteins were found to contain mainly core type 2 O-glycans with one or two sialic acid residues (shown in grey and orange symbols, respectively, FIG. 6A). Sialylated core type 1 O--glycans were also identified albeit at very low abundance (<0.5%). Interestingly, the most anionic and bioactive MonoQ CD52 fractions of CD52 (F48 and F49) had a considerably higher O-glycan occupancy (15-20%, compared to 4% in non-active fractions). Extracted ion chromatograms (EIC) of the bioactive fractions (F48 and F49) showed an absence of sialo-isomers for the most abundant O-glycan structure m/z 665.22-(GalNAc1 GlcNAc1 Gal2NeuAc2), but not for m/z 1040.41-(GalNAc1 GlcNAc1 Gal2NeuAc) (FIG. 6B). Finally, 0-glycan site localisation was determined by electron transfer/higher-energy collision dissociation (EThcD), which provided c and z ions supporting the conclusion that di-sialylated O-glycans were conjugated to Ser12, and possibly Ser10 due to lack of diagnostic ions, whereas the mono-sialylated O-glycans were only found on Thr8 (FIG. 6C).

Table 2: Sialic acid content and antennae distribution of recombinant human CD52 fractions separated by anion chromatography.

(A) (upper panel) The total number of sialic acid residues and (B) (lower panel) The antennae distribution identified on CD52 fractions (F30, F46, F47, F49, F50 and F51) using PGC-LC-MS/MS.

It will be understood that the invention disclosed and defined in this specification extends to all alternative combinations of two or more of the individual features mentioned or evident from the text or drawings. All of these different combinations constitute various alternative aspects of the invention.

### REFERENCES

Bandala-Sanchez et al. (2013) Nat. Immunol. 14: 741-748.
Bandala-Sanchez et al. WO 2013/071355
Cooper et al. (2001) Proteomics 1: 340-349.
Everest-Dass et al. (2013) Journal of the American Society for Mass Spectrometry 24: 895-906.
Harvey et al. (2008) Analytical Biochemistry 376:44-60.
Jensen et al. (2012) Nature Protocols 7:1299.
Paulson and Rogers (1987) Methods in Enzymology 138. Elsevier, p. 162-168.
Schmidt and Skerra (2007) Nat. Protoc. 2: 1528-1535.
Schröter et al (1999) J. Biol. Chem. 274: 29862-29873.
Treumann et al. (1995) J. Biol. Chem. 270: 6088-6099.
Wang and Cummings (1988) Journal of Biological Chemistry 263: 4576-4585.

1.-85. (canceled)

86. A soluble CD52 glycoprotein comprising:
(i) one or more multi-antennary N-linked α-2,3-sialylated glycans selected from the group consisting of: 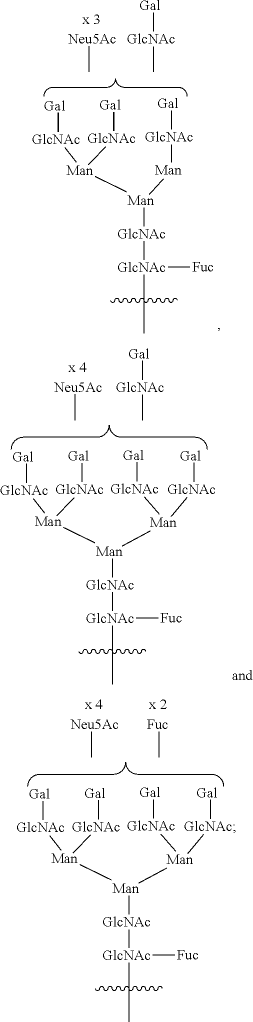
and
no N-linked bisecting GlcNAc structures; or
(ii) one or more multi-antennary N-linked α-2,3-sialylated glycans selected from the group consisting of: 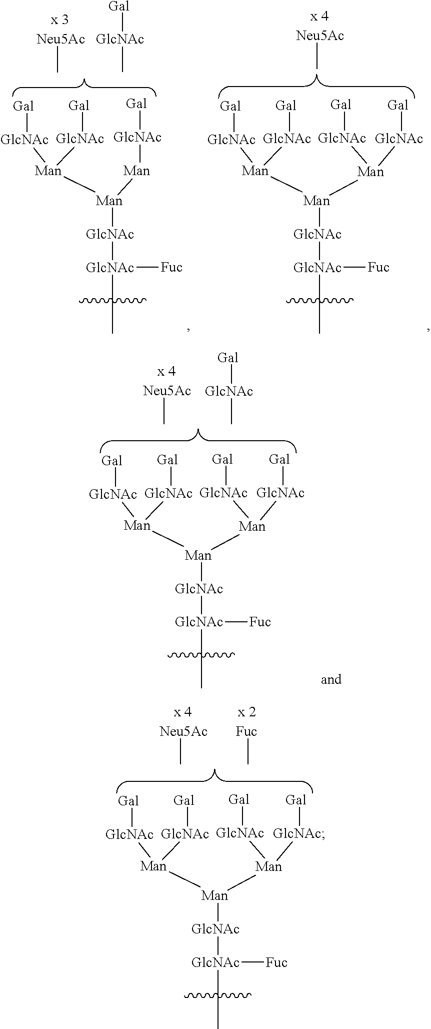
and
one or more O-glycans and no N-linked bisecting GlcNAc structures.

87. A soluble CD52 glycoprotein, comprising:
(i) one or more multi-antennary N-linked α-2,3-sialylated glycans selected from the group consisting of: 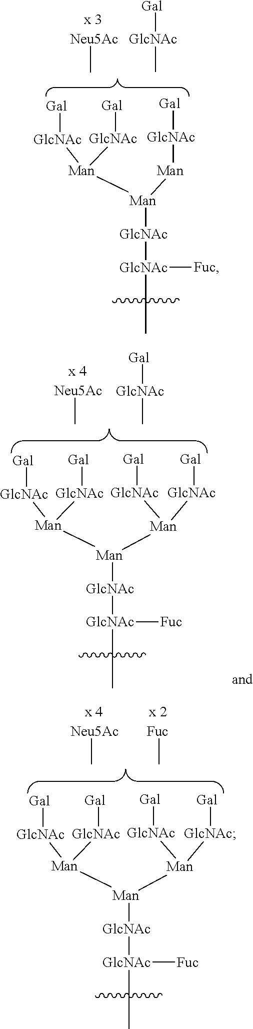
and
an isoelectric point (pl) of about 5 to about 6; or
(ii) one or more multi-antennary N-linked α-2,3-sialylated glycans selected from the group consisting of: 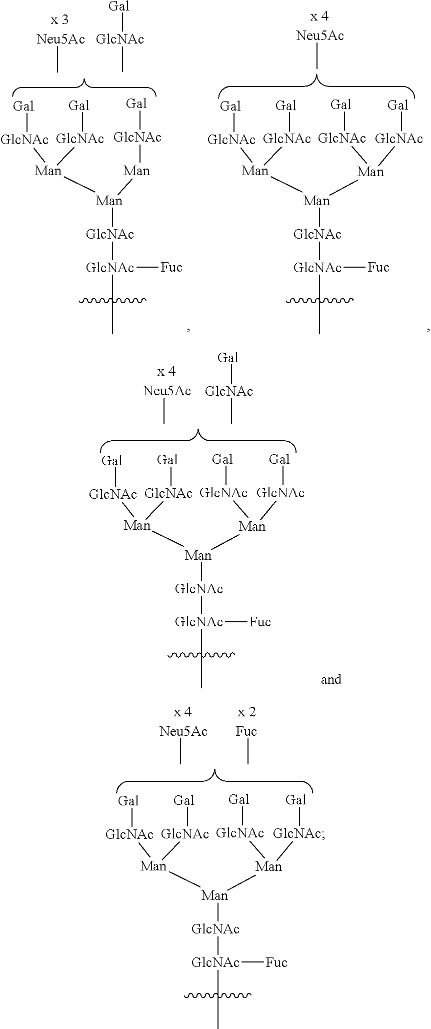
and
one or more O-glycans and an isoelectric point (pl) of about 5 to about 6.

88. The soluble CD52 glycoprotein of claim 86, comprising one or more multi-antennary N-linked α-2,3-sialylated glycans selected from the group consisting of: 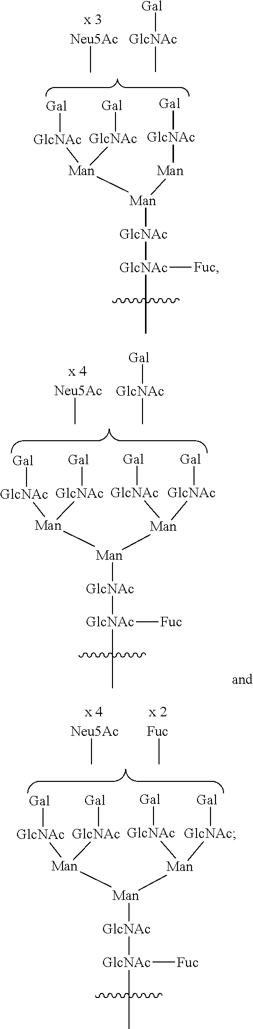
and
no N-linked bisecting GlcNAc structures.

89. The soluble CD52 glycoprotein of claim 88, wherein the one or more multi-antennary N-linked α-2,3-sialylated glycans are selected from the group consisting of:

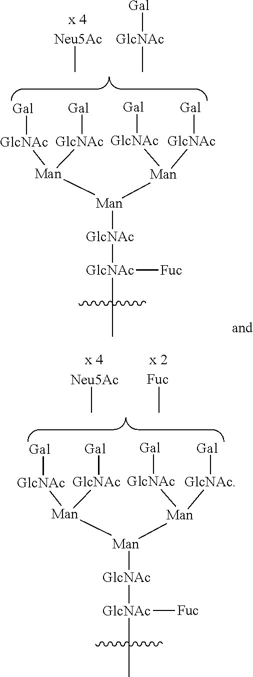

90. The soluble CD52 glycoprotein of claim 86, comprising one or more O-glycans wherein the O-glycan is a mono or di-sialylated O-glycan.

91. The soluble CD52 glycoprotein of claim 86, wherein the glycoprotein has an amino acid sequence comprising at least one amino acid suitable for N-linked glycosylation and an amino acid sequence at least 80% identical to any one or more of the amino acid sequences identified in SEQ ID NOs: 3, 4, 5, 6 or 7.

92. The soluble CD52 glycoprotein of claim 86, wherein the glycoprotein has an amino acid sequence comprising at least one amino acid suitable for N-linked glycosylation and an amino acid sequence at least 90% identical to any one or more of the amino acid sequences identified in SEQ ID NOs: 3, 4, 5, 6 or 7.

93. The soluble CD52 glycoprotein of claim 86, wherein the glycoprotein has an amino acid sequence comprising at least one amino acid suitable for N-linked glycosylation and an amino acid sequence at least 95% identical to any one or more of the amino acid sequences identified in SEQ ID NOs: 3, 4, 5, 6 or 7.

94. The soluble CD52 glycoprotein of claim 86, wherein the glycoprotein has an amino acid sequence identical to the amino acid sequences identified in SEQ ID NOs: 3, 4, 5, 6 or 7.

95. The soluble CD52 glycoprotein of claim 86, wherein the glycoprotein has an amino acid sequence identical to the amino acid sequence identified in SEQ ID NO: 3.

96. The soluble CD52 glycoprotein of claim 95, wherein the N-glycan is linked to the asparagine (N) residue 3.

97. The soluble CD52 glycoprotein of claim 90, wherein the glycoprotein has an amino acid sequence identical to the amino acid sequence identified in SEQ ID NO: 3 and the one or more O-glycans are linked to serine (S) residue 12, serine residue (S) 10, and/or threonine (T) residue 8.

98. The soluble CD52 glycoprotein of claim 90, wherein the glycoprotein is a di-sialylated O-glycan and the glycoprotein has an amino acid sequence identical to the amino acid sequence identified in SEQ ID NO: 3 and the O-glycans are linked to serine (S) residue 12 and/or serine residue (S) 10.

99. A fusion protein comprising the soluble CD52 glycoprotein of claim 86 conjugated with a second protein.

100. The fusion protein of claim 99, wherein the second protein is an antibody fragment.

101. The fusion protein of claim 99, wherein the second protein is a purification tag.

102. The fusion protein of claim 101, wherein the second protein is a purification tag and the purification tag is selected from the group consisting of a His tag, T7 tag, FLAG tag, S-tag, HA tag, c-Myc tag, DHFR, a chitin biding domain, a calmodulin binding domain, a cellulose binding domain and a Strep 2 tag.

103. A composition comprising:
the soluble CD52 glycoprotein of claim 86; and
serum.

104. The composition of claim 103, further comprising insulin and/or an autoantigen.

105. A method of suppressing effector T-cell function and/or immune response comprising administration of a therapeutically effective amount of the CD52 glycoprotein of claim 86 to a subject in need thereof.

106. A method of treating or preventing a disease or condition mediated by effector T-cell function, inflammation, or sepsis comprising administration of a therapeutically effective amount of the CD52 glycoprotein of claim 86 to a subject in need thereof.
